SEQUENCE LISTING (32 sequences)

SEQ ID NO: 1 - protein, 747 aa, Homo sapien
MADEAALALQPGGSPSAAGADREAASSPAGEPLRKRPRRDGPGLERSPGEPGGAAPEREVPAAARGCPGAAAAALWREAEAEAAAAGGEQEAQATAAAGEGDNGPGLQGPSREPPLADNLYDEDDDDEGEEEEEAAAAAIGYRDNLLFGDEIITNGFHSCESDEEDRASHASSSDWTPRPRIGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRTQKELAYLSELPPTPLHVSEDSSSPERTSPPDSSVIVTLLDQAAKSNDDLDVSESKGCMEEKPQEVQTSRNVESIAEQMENPDLKNVGSSTGEKNERTSVAGTVRKCWPNRVAKEQISRRLDGNQYLFLPPNRYIFHGAEVYSDSEDDVLSSSSCGSNSDSGTCQSPSLEEPMEDESEIEEFYNGLEDEPDVPERAGGAGFGTDGDDQEAINEAISVKQEVTDMNYPSNKS

SEQ ID NO: 2 - protein, 44 aa, Homo sapien
QIVINILSEPPKRKKRKDINTIEDAVKLLQECKKIIVLTGAGVS

SEQ ID NO: 3 - protein, 22 aa, Homo sapien
QIVINILSEPPKRKKRKDINTI

SEQ ID NO: 4 - protein, 823 aa, Drosophila melanogaster
MMENYEEIRLGHIRSKDLGNQVPDTTQFYPPTKFDFGAEILASTSTEAEAEATATTTEPATSELAGKANGEIKTKTLAAREEQEIGANLEHKTKNPTKSMGEDEDDEEEEEEDDEEEEEDDEEGITGTSNEDEDSSSNCSSSVEPDWKLRWLQREFYTGRVPRQVIASIMPHFATGLAGDTDDSVLWDYLAHLLNEPKRRNKLASVNTFDDVISLVKKSQKIIVLTGAGVSVSCGIPDFRSTNGIYARLAHDFPDLPDPQAMFDINYFKRDPRPFYKFAREIYPGEFQFQPSPCHRFIKMLETKGKLLRNYTQNIDTLERVAGIQRVIECHGSFSTASCTKCRFKCNADALRADIFAQRIPVCPQCQPNKEQSVDASVAVTEEELRQLVENGIMKPDIVFFGEGLPDEYHTVMATDKDVCDLLIVIGSSLKVRPVAHIPSSIPATVPQILINREQLHHLKFDVELLGDSDVIINQICHRLSDNDDCWRQLCCDESVLTESKELMPPEHSNHHLHHHLHHHRHCSSESERQSQLDTDTQSIKSNSSADYILGSAGTCSDSGFESSTFSCGKRSTAAEAAAIERIKTDILVELNETTALSCDRLGLEGPQTTVESYRHLSIDSSKDSGIEQCDNEATPSYVRPSNLVQETKTVAPSLTPIPQQRGKRQTAAERLQPGTFYSHTNNYSYVFPGAQVFWDNDYSDDDDEEEERSHNRHSDLFGNVGHNYKDDDEDACDLNAVPLSPLLPHSLEAHIFTDIVNGSNEPLPNSSPGQKRTACIIEQQPTPAIETEIPPLKKRRPSEENKQQTQIERSEESPPPGQLAAV

SEQ ID NO: 5 - protein, 44 aa, Drosophila melanogaster
DYLAHLLNEPKRRNKLASVNTFDDVISLVKKSQKIIVLTGAGVS

SEQ ID NO: 6 - protein, 22 aa, Drosophila melanogaster
DYLAHLLNEPKRRNKLASVNTF

SEQ ID NO: 7 - protein, 607 aa, Caenorhabditis elegans
MSRDSGNDSEVAVTHGEVQEITEENPEIGSMHITQETDISDAPETNTDSSRQRTESTTSVSSESWQNNDEMMSNLRRAQRLLDDGATPLQIIQQIFPDFNASRIATMSENAHFAILSDLLERAPVRQKLTNYNSLADAVELFKTKKHILVLTGAGVSVSCGIPDFRSKDGIYARLRSEFPDLPDPTAMFDIRYFRENPAPFYNFAREIFPGQFVPSVSHRFIKELETSGRLLRNYTQNIDTLEHQTGIKRVVECHGSFSKCTCTRCGQKYDGNEIREEVLAMRVAHCKRCEGVIKPNIVFFGEDLGREFHQHVTEDKHKVDLIVVIGSSLKVRPVALIPHCVDKNVPQILINRESLPHYNADIELLGNCDDIIRDICFSLGGSFTELITSYDSIMEQQGKTKSQKPSQNKRQLISQEDFLNICMKEKRNDDSSDEPTLKKPRMSVADDSMDSEKNNFQEIQKHKSEDDDDTRNSDDILKKIKHPRLLSITEMLHDNKCVAISAHQTVFPGAECSFDLETLKLVRDVHHETHCESSCGSSCSSNADSEANQLSRAQSLDDFVLSDEDRKNTIHLDLQRADSCDGDFQYELSETIDPETFSHLCEEMRI

SEQ ID NO: 8 - protein, 44 aa, Caenorhabditis elegans
AILSDLLERAPVRQKLTNYNSLADAVELFKTKKHILVLTGAGVS

SEQ ID NO: 9 - protein, 22 aa, Caenorhabditis elegans
AILSDLLERAPVRQKLTNYNSL

SEQ ID NO: 10 - protein, 562 aa, Saccharomyces cerevisiae
MTIPHMKYAVSKTSENKVSNTVSPTQDKDAIRKQPDDIINNDEPSHKKIKVAQPDSLRETNTTDPLGHTKAALGEVASMELKPTNDMDPLAVSAASVVSMSNDVLKPETPKGPIIISKNPSNGIFYGPSFTKRESLNARMFLKYYGAHKFLDTYLPEDLNSLYIYYLIKLLGFEVKDQALIGTINSIVHINSQERVQDLGSAISVTNVEDPLAKKQTVRLIKDLQRAINKVLCTRLRLSNFFTIDHFIQKLHTARKILVLTGAGVSTSLGIPDFRSSEGFYSKIKHLGLDDPQDVFNYNIFMHDPSVFYNIANMVLPPEKIYSPLHSFIKMLQMKGKLLRNYTQNIDNLESYAGISTDKLVQCHGSFATATCVTCHWNLPGERIFNKIRNLELPLCPYCYKKRREYFPEGYNNKVGVAASQGSMSERPPYILNSYGVLKPDITFFGEALPNKFHKSIREDILECDLLICIGTSLKVAPVSEIVNMVPSHVPQVLINRDPVKHAEFDLSLLGYCDDIAAMVAQKCGWTIPHKKWNDLKNKNFKCQEKDKGVYVVTSDEHPKTL

SEQ ID NO: 11 - protein, 44 aa, Saccharomyces cerevisiae
DLQRAINKVLCTRLRLSNFFTIDHFIQKLHTARKILVLTGAGVS

SEQ ID NO: 12 - protein, 22 aa, Saccharomyces cerevisiae
DLQRAINKVLCTRLRLSNFFTI

SEQ ID NO: 13 - protein, 355 aa, Homo sapien
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMEHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 14 - protein, 44 aa, Homo sapien
ADKGKCGLPEIFDPPEELERKVWELARLVWQSSSVVFHTGAGIS

SEQ ID NO: 15 - protein, 400 aa, Homo sapien
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 16 - protein, 44 aa, Homo sapien
REGLKRRQEEVCDDPEELRGKVRELASAVRNAKYLVVYTGAGIS

SEQ ID NO: 17 - protein, 389 aa, Homo sapien
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 18 - protein, 44 aa, Homo sapien
FSQTLSLGSQKERLLDELTLEGVARYMQSERCRRVICLVGAGIS

SEQ ID NO: 19 - protein, 399 aa, Homo sapien
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 20 - protein, 44 aa, Homo sapien
VGASSVVGSGGSSDKGKLSLQDVAELIRARACQRVVVMVGAGIS

SEQ ID NO: 21 - protein, 314 aa, Homo sapien
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 22 - protein, 44 aa, Homo sapien
PCSKASIGLFVPASPPLDPEKVKELQRFITLSKRLLVMTGAGIS

SEQ ID NO: 23 - protein, 310 aa, Homo sapien
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 24 - protein, 44 aa, Homo sapien
LKPPASTRNQICLKMARPSSSMADFRKFFAKAKHIVIISGAGVS

SEQ ID NO: 25 - protein, 5 aa, Artificial sequence
  Synthetic
KGGAK

SEQ ID NO: 26 - protein, 4 aa, Artificial sequence
  Synthetic
RHKK

SEQ ID NO: 27 - protein, 4 aa, Artificial sequence
  Synthetic
RHKK

SEQ ID NO: 28 - protein, 4 aa, Artificial sequence
  Synthetic
QPKK

SEQ ID NO: 29 - protein, 5 aa, Artificial sequence
  Synthetic
DKVQK

SEQ ID NO: 30 - protein, 5 aa, Artificial sequence
  Synthetic
DKVQK

SEQ ID NO: 31 - protein, 5 aa, Artificial sequence
  Synthetic
YETFK

SEQ ID NO: 32 - protein, 5 aa, Artificial sequence
  Synthetic
KGLLK

(19) United States
(12) Patent Application Publication
(10) Pub. No.: US 2012/0172340 A1
(43) Pub. Date: Jul. 5, 2012

(54) Compositions and Methods for Selectively Activating Human Sirtuins

(75) Inventors: Konrad T. Howitz, Allentown, PA (US); Robert E. Zipkin, Wynnewood, PA (US)

(73) Assignee: BIOMOL International L.P., Plymouth Meeting, PA (US)

(21) Appl. No.: 12/900,073

(22) Filed: Oct. 7, 2010

Related U.S. Application Data
(63) Continuation of application No. 11/166,892, filed on Jun. 24, 2005, now abandoned.
(60) Provisional application No. 60/584,943, filed on Jun. 30, 2004.

(51) Int. Cl. A61K 31/655; C12N 9/99; C12N 9/78; A61K 31/519; A61K 31/435; A61K 31/35; A61K 31/235; A61K 31/185; A61K 31/16; A61K 31/12; A61K 31/05; A61K 31/04; A61P 3/10; A61P 3/06; C12Q 1/34

(52) U.S. Cl. 514/151; 435/18; 435/184; 435/227; 514/262.1; 514/277; 514/456; 514/544; 514/576; 514/616; 514/630; 514/689; 514/733; 514/740

(57) ABSTRACT

Methods for identifying selective activators of SIRT5 and/or SIRT1 and methods for using these selective activators in the modulation of SIRT5 and/or SIRT1 are provided.

This patent application is a continuation of U.S. application Ser. No. 11/166,892, filed Jun. 24, 2005, which claims the benefit of priority from U.S. Provisional Application Ser. No. 60/584,943, filed Jun. 30, 2004, each of which is herein incorporated by reference in their entirety.

### BACKGROUND OF THE INVENTION

The sirtuin enzymes, also known as class III histone deactylases or HDACs, catalyze a reaction which couples deacetylation of protein ε-acetyllysine residues to the formation of O-acetyl-ADP-ribose and nicotinamide from the oxidized form of nicotinamide adenine dinucleotide or NAD⁺ (Imai, S et al. Nature 403, 795-800 (2000); Tanner, K. G. et al. Proc. Natl. Acad. Sci. USA 97, 14178-14182 (2000); Tanny, J. C. and Moazed, D. Proc. Natl. Acad. Sci. USA 98, 415-420 (2001)). Sirtuin homologs are found in all forms of life, including the archaea, the bacteria and both unicellular and multicellular eukaryotes (Smith, J. S. et al. Proc. Natl. Acad. Sci. USA 97, 6658-6663 (2000); Blander, G. and Guarente, L. Annu. Rev. Biochem. 73, 417-435 (2004); Buck, S. W. et al. J. Leukoc. Biol. 75, 1-12 (2004); and Frye, R. A. Biochem. Biophys. Res. Commun. 273, 793-798 (2000)). The founding exemplar of the group, Sir2 from baker's yeast (Saccharomyces cerevisiae), was named for its role in gene-silencing (Silent information regulator 2; Rusche, L. et al. Annu. Rev. Biochem. 72, 481-516 (2003)). Transcriptional silencing by Sir2 is linked to its deacetylation of lysines in the N-terminal tails of the histones in chromatin, hence the classification as a class III HDAC. Lysine deacetylation by sirtuins, however, extends beyond histones. Targets of sirtuin regulatory deacetylation include mammalian transcription factors such as p53 (Luo, J. et al. Cell 107, 137-48 (2001); Vaziri, H. et al. Cell 107, 149-59 (2001); Langley E. et al. EMBO J. 21, 2383-2396 (2002)), the cytoskeletal protein, tubulin (North, B. J. et al. Molecular Cell 11, 437-444 (2003)) and the bacterial enzyme, acetyl-CoA synthetase (Starai, V. J. et al. Science 298, 2390-2392 (2002); Zhao, K. et al. J. Mol. Biol. 337, 731-741 (2004). Sir2 and its closest eukaryotic homologs have a role in conserved pathways of stress-response and longevity regulation (Kenyon, C. Cell 105, 165-168 (2001); Guarente, L. and Kenyon, C. Nature 408, 255-62 (2000)). For example, yeast Sir2 is required for the lifespan extension conferred by calorie restriction and other mild stresses (Lin, S. J. et al. Science 289, 2126-8 (2000); Anderson, R. M. et al. Nature 423, 181-5 (2003)). Extra copies of the gene for Sir2 in yeast or of its homolog Sir2.1 in the nematode worm C. elegans, have also been demonstrated to extend lifespan by 30-70% and approximately 50%, respectively (Tissenbaum, H. A. and Guarente, L. Nature 410, 227-30 (2001)). Further, C. elegans Sir2.1 functions in the insulin/IGF-1 signaling pathway (Kenyon, C. Cell 105, 165-168 (2001); Guarente, L. and Kenyon, C. Nature 408, 255-62 (2000)), a pathway that has also been shown to regulate lifespan in rodents (Holzenberger, M. et al. Nature 421, 182-187 (2003); Bluher, M. et al. Science 299, 489-490 (2003)). SIRT1, the closest human homolog to Sir2 and Sir2.1 has recently been shown to also act in the insulin/IGF-1 pathway, via its regulation of FOXO transcription factors (Motta, M. C. et al. Cell 116, 551-563 (2004); Brunet, A. et al. Science 303, 2011-2015 (2004); Van Der Horst, A. et al. J. Biol. Chem. 279, 28873-28879 (2004)).

Phylogenetic analysis of the conserved domains of sixty prokaryotic and eukaryotic sirtuins resulted in an unrooted tree comprising five main homology groups (classes I, II, III, IV and V; Frye, R. A. Biochem. Biophys. Res. Commun. 273, 793-798 (2000)). All yeast sirtuins fall into class I, a group further divided into subclasses a, b and c. Yeast Sir2 and other sirtuins implicated in longevity and/or insulin/IGF-1 signaling (human SIRT1, C. elegans Sir2.1 and D. melanogaster dSir2) are all part of class Ia. Class III sirtuins include archaeal, bacterial and some eukaryotic enzymes, including human SIRT5. Salmonella and E. coli “CobB” enzymes, bacterial class III sirtuins, activate acetyl-CoA synthetase by deacetylation of a lysine residue that lies within a sequence motif conserved among a variety AMP-forming enzymes, including human acetyl-CoA synthetases (Starai, V. J. et al. Science 298, 2390-2392 (2002); Luong, A. et al. J. Biol. Chem. 275, 26458-26466 (2000); Fujino, T. et al. J. Biol. Chem. 276, 11420-11426 (2001)).

There are seven identified human sirtuins (Frye, R. A. Biochem. Biophys. Res. Commun. 273, 793-798 (2000)). Of these, SIRTs 1, 2 and 3 have received the majority of the experimental attention. SIRT1, the human Sir2 homolog, is located in the nucleus and has been shown to deacetylate the transcription factors p53 (Luo, J. et al. Cell 107, 137-48 (2001); Vaziri, H. et al. Cell 107, 149-59. (2001); E. Langley et al. EMBO J. 21, 2383-2396 (2002)) and FOXOs 1, 3 and 4 (Motta, M. C. et al. Cell 116, 551-563 (2004); Brunet, A. et al. Science 303, 2011-2015 (2004); Van Der Horst, A. et al. J. Biol. Chem. 279, 28873-28879 (2004)), the histone acetyltransferase, p300 (Motta, M. C. et al. Cell 116, 551-563 (2004)) and the H3/H4 histones (Senawong, T. et al. J. Biol. Chem. 278, 43041-43050 (2003)). SIRT2, which is primarily cytoplasmic, forms a complex with HDAC6 and has been shown to function as a tubulin deacetylase (North, B. J. et al. Molecular Cell 11, 437-444 (2003)). SIRT3, which is located in the mitochondria (Schwer, B. et al. J. Cell Biol. 158, 647-657 (2002); Onyango, P. et al. Proc. Natl. Acad. Sci. USA 99, 13653-13658 (2002)) is synthesized with an N-terminal targeting sequence that is removed upon mitochondrial import (Schwer, B. et al. J. Cell Biol. 158, 647-657 (2002). Although this mature, proteolytically processed form of SIRT3 has deacetylase activity in vitro (Schwer, B. et al. J. Cell Biol. 158, 647-657 (2002)), nothing else is known about SIRT3 function or its native acetylated substrates. Sequence analysis programs (MitoProt (Claros, M. G. and Vincens, P. Eur. J. Biochem. 241, 779-786 (1996)), TargetP (Emanuelsson, O. et al. J. Mol. Biol. 300, 1005-1016 (2000)) predict that SIRTs 4, 5 and 7 also should be imported mitochondrial proteins. These targeting prediction algorithms are 89.4% (MitoProt; Claros, M. G. and Vincens, P. Eur. J. Biochem. 241, 779-786 (1996)) and 90% (TargetP; Emanuelsson, O. et al. J. Mol. Biol. 300, 1005-1016 (2000)) accurate for non-plant proteins and the predictions of both have proven correct with respect to the experimentally verified localizations of the SIRTs 1, 2 and 3.

Selected plant polyphenols were recently identified as activators of SIRT1, with resveratrol, the most potent of these activators, extending the lifespans of yeast (Howitz, K. T. et al. Nature 425, 191-196 (2003)), fruit flies (D. melanogaster) and nematode worms (C. elegans)(Wood, J. G. et al. Nature 440, 686-689 (2004)).

### SUMMARY OF THE INVENTION

Small-molecule activators and inhibitors of human SIRT5, a class III sirtuin have now been identified.

Identified human SIRT5 activators include, but are not limited to, polyphenol compounds, such as plant polyphenols or analogs or derivatives thereof, selected from the group consisting of stilbenes, chalcones, and flavones and non-polyphenol dipyridamole compounds, as well as analogs or derivatives thereof. Exemplary human SIRT5 activators of the present invention are set forth herein as Formulas 1-12. Exemplary embodiments of human SIRT5 activators of the present invention activating SIRT5 activity by at least 2-fold as compared to controls include, but are not limited to, 3,5-dihydroxy-4′-chloro-trans-stilbene, dipyridamole, 3,5-dihydroxy-4′ ethyl-trans-stilbene, 3,5-dihydroxy-4′-isopropyl-trans-stilbene, 3,5-dihydroxy-4′-methyl-trans-stilbene, resveratrol, 3,5-dihydroxy-4′ thiomethyl-trans-stilbene, 3,5-dihydroxy-4′-carbomethoxy-trans-stilbene, isoliquiritgenin, 3,5-dihydro-4′ nitro-trans-stilbene, 3,5-dihydroxy-4′ azido-trans-stilbene, piceatannol, 3-methoxy-5-hydroxy-4′ acetamido-trans-stilbene, 3,5-dihydroxy-4′ acetoxy-trans-stilbene, pinosylvin, fisetin, (E)-1-(3,5-dihydrophenyl)-2-(4-pyridyl)ethene, (E)-1-(3,5-dihydrophenyl)-2-(2-napthyl)ethene, 3,5-dihydroxy-4′-acetamide-trans-stilbene, butein, quercetin, 3,5-dihydroxy-4′-thioethyl-trans-stilbene), 3,5-dihydroxy-4′ carboxy-trans-stilbene, and 3,4′-dihydroxy-5-acetoxy-trans-stilbene, and analogs and derivatives thereof. These compounds are referred to generally herein as human SIRT5 activators or human SIRT5 activating compounds.

Identified human SIRT5 inhibitors include, but are not limited to, 3-hydroxy-trans-stilbene, 4-methoxy-trans-stilbene, ZM 336372 (N-[5-(3-dimethylaminobenzamido)-2-methylphenyl]-4-hydroxybenzamide), and 3,4-dihydroxy-trans-stilbene, depicted herein in Formulas 13 through 16, respectively. These compounds are referred to generally herein as human SIRT5 inhibitors or human SIRT5 inhibiting compounds.

One aspect of the present invention relates to a method for identifying compounds as selective activators or inhibitors of human SIRT5 or human SIRT1, or alternatively as general activators or inhibitors of sirtuins including, but not limited to, human SIRT5 and human SIRT1. For example, using this method of the present invention, dipyridamole and BML-237 (3,5-dihydroxy-4′-carbomethoxy-trans-stilbene) have been identified as selective activators of SIRT5 as compared to SIRT1; BML-217 (3,5-dihydroxy-4′-chloro-trans-stilbene) has been identified as a potent activator of SIRT5 and SIRT1; and BML-243 (3,5-dihydroxy-4′-thioethyl-trans-stilbene), butein and ZM336372 have been identified as selective activators of SIRT1 as compared to SIRT5.

Another aspect of the present invention relates to a method for modulating human SIRT5 activity which comprises contacting human SIRT5 with a human SIRT5 activating or inhibiting compound identified herein. Human SIRT5 activating compounds used in this method may be selected based upon their ability to activate SIRT5 selectively or upon their ability to activate multiple classes of sirtuins.

Another aspect of the present invention relates to a method for selectively activating human SIRT1 activity by contacting SIRT1 with a compound identified in accordance with methods described herein to selectively activate human SIRT1 as compared to human SIRT5.

Another aspect of the present invention relates to a method for modulating mitochondrial acetyl-CoA synthetase (AceS2) activity in cells which comprises contacting the cells with a human SIRT5 activating compound or a human SIRT5 inhibiting compound.

Another aspect of the present invention relates to pharmaceutical compositions comprising a human SIRT5 activating compound and methods for their use as lipid-lowering agents. Such agents are expected to be useful in treatment of patients with hyperlipidemia and hyper-cholesterolemia as well as prevention and treatment of type 2 diabetes in patients.

### BRIEF DESCRIPTION OF THE FIGURES

FIG. 1A through 1C shows dose-response curves of class Ia and class Ib sirtuins to resveratrol. Initial rates of fluorogenic peptide deacetylation were determined as described by Howitz, K. T. et al. (Nature 425, 191-196 (2003)) with recombinant sirtuins expressed and purified from E. coli. FIG. 1A shows the initial rates of human SIRT1 and the E230K mutant SIRT1 determined at 37° C., with 25 μM NAD⁺ and 25 μM p53-382 peptide (BIOMOL Cat. #KI-177) as substrates. Rates for human SIRTs 2 and 3 were determined identically, except that 25 μM p53-320 (BIOMOL Cat. #KI-179) was used as the acetylated peptide substrate. FIG. 1 B shows initial rates for ySir2 determined at 30° C. with 200 μM NAD⁺ and 200 μM p53-382. Rates for Sir2.1 and dSir2 were determined at 25° C. with 50 μM NAD⁺ and 50 μM “Fluor de Lys” acetylated lysine substrate (BIOMOL Cat. #KI-104). FIG. 1C shows data from FIG. 1B replotted with an expanded x-axis ([Resveratrol], μM) in order to better display the resveratrol stimulation of ySir2 at low concentrations.

FIGS. 2A and 2B show a SIRT1 mutation affecting resveratrol activation (E230K) occurring in a stretch of sequence conserved within class Ia sirtuins. FIG. 2A shows forty-four residues inclusive of the N-terminal and a conserved GAG(I/V)S motif in seven known human sirtuins aligned with the ClustalW program (Thompson, J. D. Nucl. Acids Res. 22, 4673-4680 (1994)). Sequences are shown in single-letter amino acid code and the SIRT1 E230 is underlined. Residue number of the final S in the GAG(I/V)S motif is shown to the right of each sequence. FIG. 2B shows alignment by ClustalW of the first 22 residues of the class Ia sequences in FIG. 2A. SIRT1 E230 is again shown underlined. Key to residue relationship: Bold-identical residue, italics=strong homology, lower case=weak homology. Aligned sequences were obtained at the following Genbank accession numbers—SIRT1: NM_{—}012238 (full length sequence set forth in SEQ ID NO:1; forty-four residue fragment of FIG. 2A set forth in SEQ ID NO:2; twenty-two residue fragment of FIG. 2B set forth in SEQ ID NO:3), dSir2: AF068758 (full length sequence set forth in SEQ ID NO:4; forty-four residue fragment of FIG. 2A set forth in SEQ ID NO:5; twenty-two residue fragment of FIG. 2B set forth in SEQ ID NO:6), Sir2.1: NM_{—}069511 (full length sequence set forth in SEQ ID NO:7; forty-four residue fragment of FIG. 2A set forth in SEQ ID NO:8; twenty-two residue fragment of FIG. 2B set forth in SEQ ID NO:9), Sir2: NC_{—}001136 (full length sequence set forth in SEQ ID NO:10; forty-four residue fragment of FIG. 2A set forth in SEQ ID NO:11; twenty-two residue fragment of FIG. 2B set forth in SEQ ID NO:12), SIRT6: NM_{—}016539 (full length sequence set forth in SEQ ID NO:13; forty-four residue fragment of FIG. 2A set forth in SEQ ID NO:14), SIRT7: NM_{—}016538 (full length sequence set forth in SEQ ID NO:15; forty-four residue fragment of FIG. 2A set forth in SEQ ID NO:16), SIRT2: NM_{—}012237 (full length sequence set forth in SEQ ID NO:17; forty-four residue fragment of FIG. 2A set forth in SEQ ID NO:18), SIRT3: NM_{—}012239 (full length sequence set forth in SEQ ID NO:19; forty-four residue fragment of FIG. 2A set forth in SEQ ID NO:20), SIRT4: NM_{—}012240 (full length sequence set forth in SEQ ID NO:21; forty-four residue fragment of FIG. 2A set forth in SEQ ID NO:22), and SIRT5: NM_{—}012241 (full length sequence set forth in SEQ ID NO:23; forty-four residue fragment of FIG. 2A set forth in SEQ ID NO:24).

FIG. 3 is a bar graph showing recombinant SIRT5 deacetylation rates (Arbitrary Fluorescent Units (AFU)/minute) for nine peptides comprising sequences from human acetylated proteins. Peptides are described in Table 1, infra.

FIG. 4A through 4C shows increases in SIRT5 activity by resveratrol resulting from alteration in substrate kinetic constants. In these experiments, the rate of p53-382 peptide deacetylation (BIOMOL Cat. #KI-177) was determined with indicated changes in substrate and resveratrol concentrations. All data points represent the mean of three determinations and error bars are the standard error of the mean. Kinetic constants in FIGS. 4B and 4C were determined by non-linear least squares fits to the Michaelis-Menten equation. FIG. 4A shows SIRT5 deacetylation rate determined with 500 μM peptide and 100 μM NAD⁺ in the presence of the indicated resveratrol concentrations. Fold-stimulation was calculated by dividing all rates by the no-resveratrol solvent control (0.1% v/v dimethylsulfoxide). FIG. 4B shows SIRT5 kinetics with respect to p53-382 concentration determined in the presence of 12 mM NAD⁺ and in the presence (open triangles) or absence (closed squares) of 500 μM resveratrol. FIG. 4C shows SIRT5 kinetics with respect to NAD⁺ concentration determined in the presence of 1 mM p53-382 peptide and in the presence (open triangles) or absence (closed squares) of 500 μM resveratrol.

FIG. 5 is a western blot which demonstrates that

SIRT5 is found in vivo, in cultured human and rat cells and mouse, rat and bovine tissues, at a lower molecular weight than those calculated for the full-length proteins encoded by its mRNA transcripts or that observed for full-length recombinant SIRT5. For these experiments, a rabbit polyclonal antibody was produced against recombinant human SIRT5 (Isoform 1; NM_{—}012241) and depleted of cross-reacting antibodies by chromatography on affinity media containing covalently bound recombinant human SIRTs 1, 2 and 3. Molecular weight markers, recombinant SIRT5 preparations, cell and tissue samples were subjected to SDS-PAGE on a 10-20% polyacrylamide gel and then transferred to a PVDF filter. The blot was blocked with 5% BSA and developed with a 1/2500 dilution of the SIRT5 antibody, a 1/2000 dilution of secondary antibody (donkey anti-rabbit IgG coupled to alkaline phosphatase, Jackson Immunoresearch) and color developed with BCIP/NBT reagent (Moss Inc.). A plot of log (MW) vs. the distance migrated by the prestained markers (far left lane) was used to calculate molecular weights for the protein bands indicated by asterisks in lanes 1-11. Lane #) Sample; calculated molecular weight(s): 1) recombinant human SIRT5 fused to 2.5 kDa His6 tag; 37.6 kDa (theoretical MW=36.0 kDa), 2) bovine heart; 28.4 kDa, 3) HeLa cell cytosolic extract (human cervical carcinoma line); 29.2 kDa, 4) PC12 cells (rat neuronal line); 30.9 & 27.6 kDa, 5) Jurkat cells (human T-cell lymphoma line); 29.2 kDa, 6) rat thymus; 29.2 & 27.6 kDa, 7) mouse brain; 27.6 kDa, 8) HL60 cells (human promonocytic line); 27.6 kDa, 9) rat liver; 27.6 kDa, 10) recombinant human SIRT5 (no His6 tag); 33.6 kDa (theoretical MW=33.9 kDa), 11) mouse liver; 25.4 kDa.

FIG. 6 is a bar graph which shows that human recombinant SIRT5 with its 39 N-terminal residues deleted (SIRT5Δ1-39) is an active deacetylase and is stimulated by resveratrol. Using either purified full-length SIRT5 or purified SIRT5Δ1-39 initial rates of p53-382 peptide deacetylation (BIOMOL Cat. #KI-177) per μg of protein were determined in the presence of 12 mM NAD⁺. Rates were determined either in absence (Control) or presence (+Resveratrol) of 500 μM resveratrol.

### DETAILED DESCRIPTION OF THE INVENTION

The present invention relates to the identification of compounds that activate or inhibit human SIRT5 and/or human SIRT1 and methods for use of such compounds in modulating human SIRT5 and/or human SIRT1 activity and enzymatic activities dependent thereon.

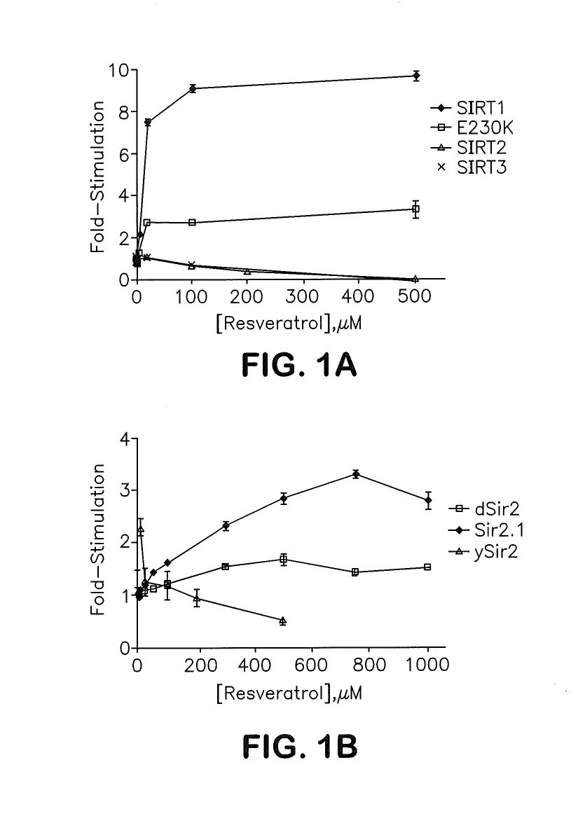

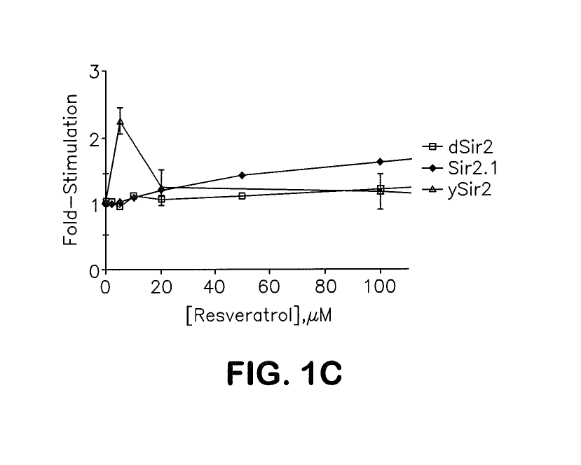

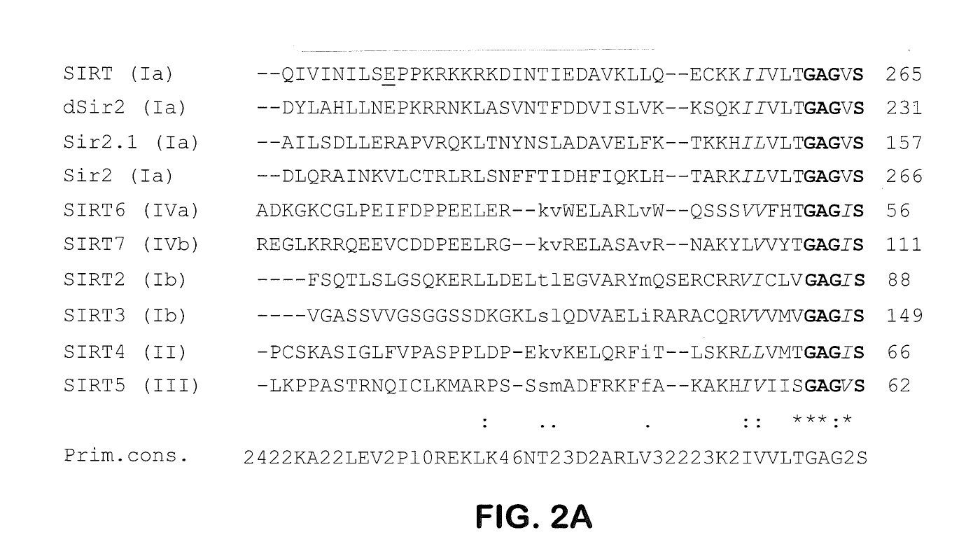

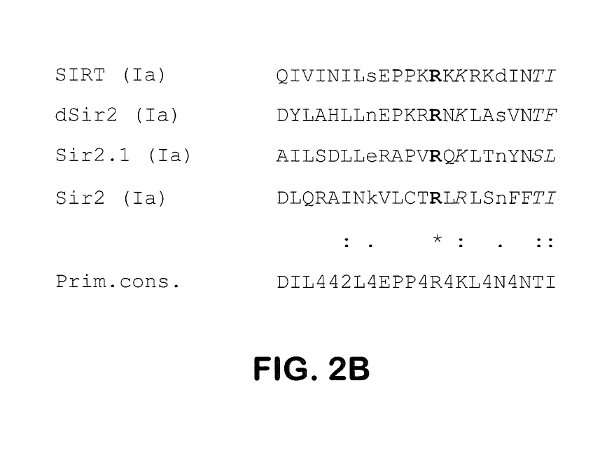

Prior to the instant invention, activation of sirtuins by compounds such a resveratrol had only been observed in the class Ia enzymes most closely related to SIRT1 (see FIG. 1). Specifically, as shown in FIG. 1, resveratrol-dependent rate increases are seen in class Ia sirtuins (human SIRT1, D. melanogaster dSir2, C. elegans Sir2.1, S. cerevisiae (budding yeast) ySir2), but not class lb sirtuins (human SIRT2 and SIRT3). Further, this specificity for class Ia sirtuins seemed to have a structural basis in that a single residue substitution (E230K) in SIRT1 that diminished resveratrol activation was located in a stretch of sequence, outside the core sirtuin domain, that only shows signs of conservation within class Ia (see FIG. 2B).

Thus, the demonstration herein that human SIRT5 is activated by resveratrol and by other polyphenols that activate SIRT1 is surprising.

Human SIRT5 was first tested for its deacetylation activity with a panel of fluorogenic, lysine-acetylated peptides patterned on acetylation sites from histone H4, and the transcription factors p53 and NF-κB p65. For these experiments, recombinant human SIRT5 (Isoform 1, Genbank Accession #NM_{—}012241 (SEQ ID NO:23)) was cloned with an N-terminal histidine tag and expressed in E. coli and then purified in accordance with procedure described for Sir2 and SIRT1 (Howitz, K. T. et al. Nature 425, 191-196 (2003)). Table 1 below sets forth the name, sequence source and sequence of the peptides used in these experiments.

TABLE 1
Fluoregenic, lysine-acetylated Peptides used to
test SIRT5 deacetylation activity

| BIOMOL | | | SEQ |
|---|---|---|---|
| Cat. # or | | | ID |
| Peptide Name | Sequence Source | Peptide Sequence | NO: |
| KI-104 | NA | K (Ac) | |
| KI-174 | Histone H4, | K-G-G-A-K (Ac) | 25 |
| | 12-16 | | |
| KI-177 | p53, 379-382 | R-H-K-K (Ac) | 26 |
| KI-178 | p53, 379-382 | R-H-K (Ac)-K(Ac) | 27 |
| KI-179 | p53, 317-320 | Q-P-K-K (Ac) | 28 |
| p65-221 | NF-κB p65 | D-K-V-Q-K (Ac) | 29 |
| | (Rel A), 217-221 | | |
| p65-221 | NF-κB p65 | D-K(Ac)-V-Q-K (Ac) | 30 |
| (diAc) | (Rel A), 217-221 | | |
| p65-310 | NF-κB p65 | Y-E-T-F-K (Ac) | 31 |
| | (Rel A), 306-310 | | |
| H4-12 | Histone H4, | K-G-L-L-K (Ac) | 32 |
| | 8-12 | | |

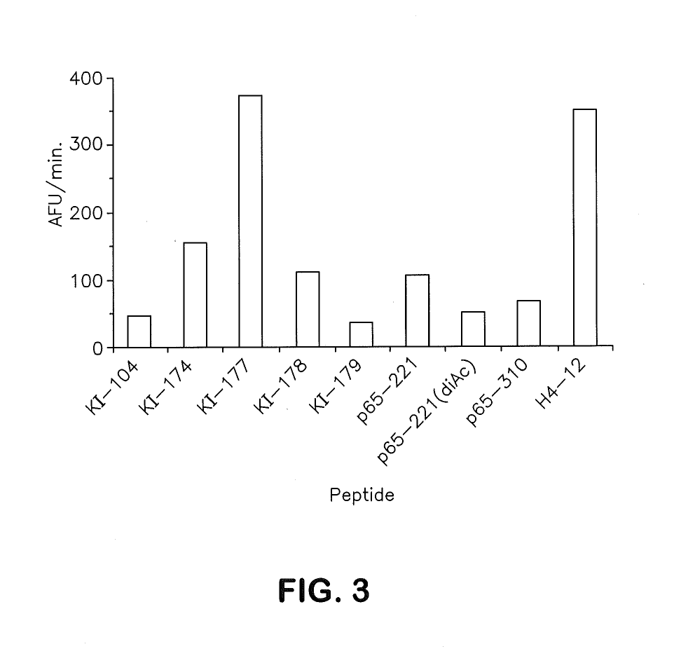

Results from these experiments are depicted in FIG. 3. As shown therein, a peptide based on the p53 lysine-382 acetylation site (BIOMOL Cat. #KI-177) was the most actively deacetylated peptide tested for SIRT1 and SIRT5.

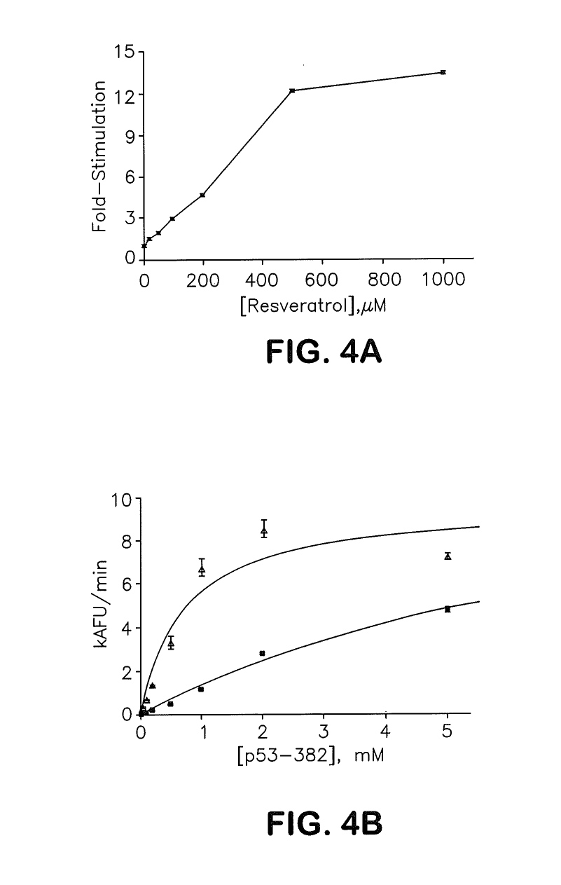

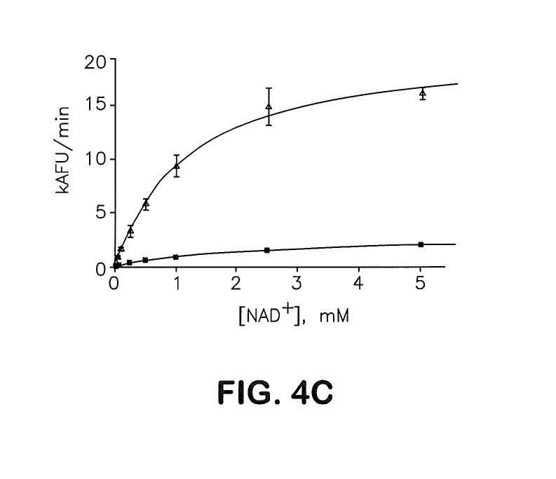

Resveratrol was also demonstrated to activate human SIRT5. A range of resveratrol concentrations was tested for their effects on the SIRT5 deacetylation rate at sub-saturating concentrations of NAD⁺ and the peptide substrate. Results from this experiment are depicted in FIG. 4A. Maximum stimulation with resveratrol was of a similar magnitude to that observed with SIRT1 as seen by comparison of FIGS. 1A and 4A. However, the maximum rate stimulation occurred at substantially higher resveratrol concentration for SIRT5 (>500 μM) than for SIRT1 (>100 μM).

One clear source of the resveratrol stimulation of human SIRT5 is an increase in the affinity of human SIRT5 for the acetylated peptide substrate in the presence of resveratrol. Vₘₐₓ for the p-53 peptide substrate (Biomol Cat. #KI-177) was 13 kAFU/minute (1000 AFU/minute) in the absence of resveratrol and 9.7 kAFU/minute in the presence of 500 μM resveratrol. Kₘ for the p53-peptide substrate was 8.9 mM in the absence of resveratrol and 0.71 mM in the presence of 500 μM resveratrol. See FIG. 4B. Thus, at a saturating level of NAD⁺ (12 mM), the addition of 500 μM resveratrol lowered the Kₘ for the p53-382 peptide by more than ten-fold (0.71 vs. 8.9 mM).

Vₘₐₓ for NAD⁺ was 3.2 kAFU/minute in the absence of resveratrol and 21 kAFU/minute in the presence of 500 μM resveratrol. Kₘ for the NAD⁺ was 2.4 mM in the absence of resveratrol and 1.2 mM in the presence of 500 μM resveratrol. See FIG. 4C. However, the NAD⁺ kinetics could not be determined under saturating peptide conditions, due to limited solubility of the peptide substrate. Therefore, while resveratrol did decrease the Kₘ for NAD⁺ when assayed at 1 mM p53-382 (see FIG. 4C), its strong apparent effect on Vₘₐₓ is due, at least in part, to the peptide Kₘ effect already noted.

A group of compounds previously shown to activate SIRT1 were also assayed for their ability to activate SIRT5 Results are shown in Table 2 and are ranked according to their activation of SIRT5. To facilitate comparison to SIRT1 data previously disclosed by Howitz, K. T. et al. (Nature 425, 191-196 (2003)), conditions yielding a similar range of activations for SIRT5 were used. Thus, for SIRT 1, conditions were as follows: 25 μM NAD⁺, 25 μM p53-382 peptide, 100 μM test compounds. The maximum stimulation observed for SIRT1 was a 12.6-fold increase in activity by BML-243 (3,5-dihydroxy-4′ thioethyl-trans-stilbene). Conditions for SIRT5 were as follows: 500 μM NAD⁺, 100 μM p53-382 peptide, 200 μM test compounds. The maximum stimulation observed for SIRT5 was a 13.6-fold increase in activity by BML-217 (3,5-dihydroxy-4′-chloro-trans-stilbene).

TABLE 2
Rate Effects of SIRT5 Activators; Comparison to
SIRT1

| | | SIRT5 | | SIRT1 | |
|---|---|---|---|---|---|
| | | Ratio of | | Ratio to | |
| | | Control | | Control | |
| | | Rates | | Rates | |
| | | Mean ± SE | | Mean ± SE | |
| | | Compounds | | Compounds | |
| Compound | Structure | at 200 μM | N | at 100 μM | N |
| BML-217 (3,5- Dihydroxy- 4′-chloro- trans- stilbene) | 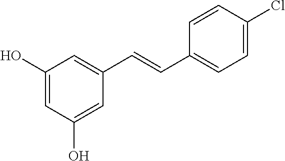 | 13.6 ± 0.4 | 3 | 10.6 ± 0.4 | 3 |
| Dipyridamole (2,6-bis (Diethanolamino)- 4,8- dipiperidino- pyrimido[5,4-d] pyrimidine) | 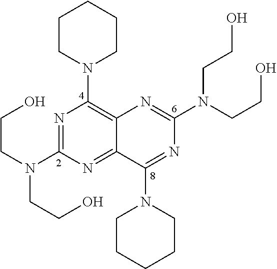 | 8.56 ± 0.30 | 3 | 3.54 ± 0.20 | 2 |
| BML-225 (3,5- Dihydroxy- 4′-ethyl- trans- stilbene) | 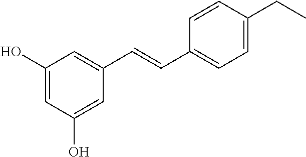 | 8.41 ± 0.36 | 3 | 9.373 ± 0.014 | 3 |
| BML-231 (3,5- Dihydroxy- 4′- isopropyl- trans- stilbene) | 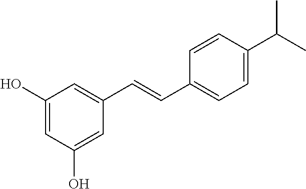 | 8.41 ± 0.22 | 3 | 6.01 ± 0.15 | 3 |
| BML-228 (3,5- Dihydroxy- 4′-methyl- trans- stilbene) | 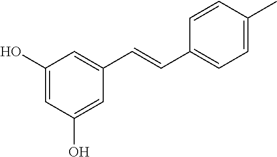 | 8.12 ± 0.31 | 3 | 7.72 ± 0.12 | 3 |
| Resveratrol (3,5,4′- Trihydroxy- trans- stilbene) | 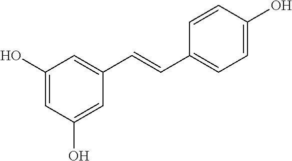 | 4.95 ± 0.48 | 15 | 10.4 ± 0.5 | 43 |
| BML-230 (3,5- Dihydroxy- 4′- thiomethyl- trans- stilbene) | 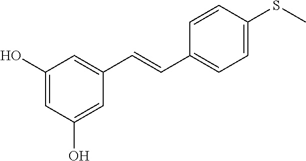 | 4.60 ± 0.17 | 3 | 6.84 ± 1.26 | 6 |
| BML-237 (3,5- Dihydroxy- 4′- carbomethoxy- trans- stilbene) | 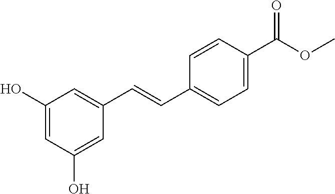 | 4.50 ± 0.28 | 3 | 2.74 ± 0.37 | 2 |
| Isoliquiritigenin (4,2′,4′- Trihydroxy- chalcone) | 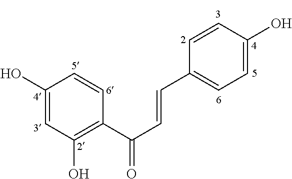 | 3.93 ± 0.30 | 3 | 7.57 ± 0.84 | 6 |
| BML-229 (3,5- Dihydroxy- 4′-nitro- trans- stilbene) | 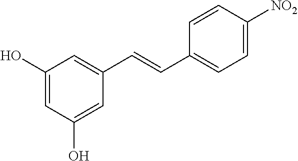 | 3.43 ± 0.10 | 3 | 6.78 ± 0.22 | 3 |
| BML-232 (3,5- Dihydroxy- 4′-azido- trans- stilbene) | 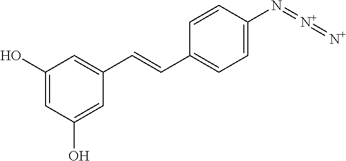 | 3.36 ± 0.14 | 3 | 7.24 ± 0.12 | 3 |
| Piceatannol (3,5,3′,4′- Tetrahydroxy- trans- stilbene) | 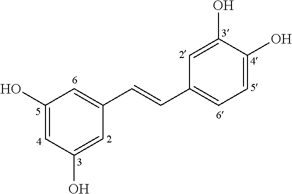 | 3.23 ± 0.21 | 3 | 7.90 ± 0.50 | 3 |
| BML-223 (3-methoxy- 5-hydroxy- 4′-acetamido- trans- stilbene) | 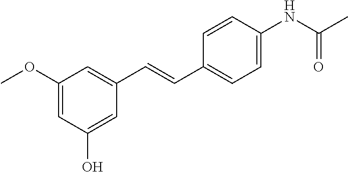 | 3.10 ± 0.016 | 3 | ND | |
| BML-221 (3,5- Dihydroxy- 4′-acetoxy- trans- stilbene) | 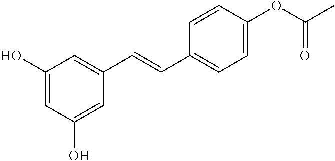 | 2.89 ± 0.15 | 3 | 3.05 ± 0.54 | 6 |
| Pinosylvin (3,5- Dihydroxy- trans- stilbene) | 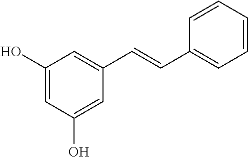 | 2.71 ± 0.092 | 3 | 9.95 ± 0.45 | 6 |
| Fisetin (3,7,3′,4′- Tetrahydroxy- flavone) | 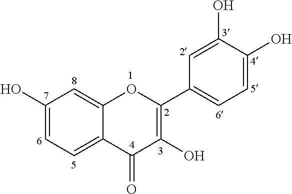 | 2.61 ± 0.19 | 3 | 6.58 ± 0.69 | 3 |
| BML-236 (E)-1-(3,5- Dihydroxyphenyl)- 2-(4-pyridyl) ethene | 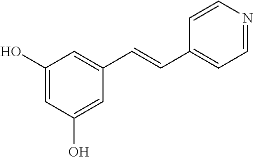 | 2.49 ± 0.29 | 3 | 1.26 ± 0.12 | 3 |
| BML-218 (E)-1-(3,5- Dihydroxyphenyl)- 2-(2-napthyl) ethene | 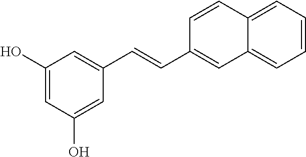 | 2.47 ± 0.22 | 3 | 3.05 ± 0.37 | 6 |
| BML-222 (3,5- Dihydroxy- 4′- acetamide- trans- stilbene) | 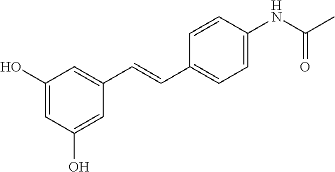 | 2.26 ± 0.17 | 3 | 1.88 ± 0.11 | 3 |
| Butein (3,4,2′,4′- Tetra- hydroxy- chalcone) | 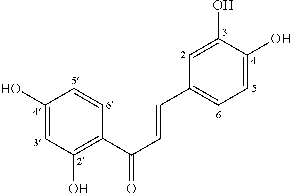 | 2.19 ± 0.10 | 3 | 8.53 ± 0.89 | 6 |
| Quercetin (3,5,7,3′,4′- Penta- hydroxyflavone) | 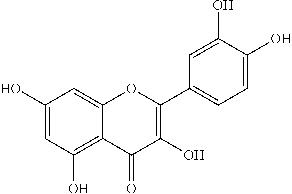 | 2.18 ± 0.10 | 3 | 4.59 ± 0.47 | 16 |
| BML-243 (3,5- Dihydroxy- 4′- thioethyl- trans- stilbene) | 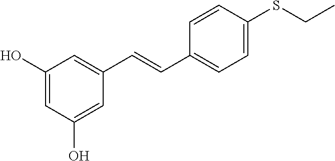 | 2.12 ± 0.34 | 3 | 12.6 ± 0.4 | 3 |
| BML-238 (3,5- Dihydroxy- 4′-carboxy- trans- stilbene) | 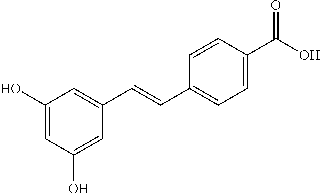 | 2.09 ± 0.25 | 3 | 1.36 ± 0.10 | 3 |
| BML-227 (3,4′- Dihydroxy- 5-acetoxy- trans- stilbene) | 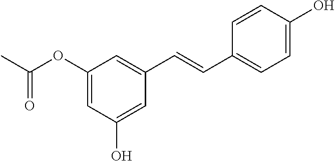 | 2.09 ± 0.25 | 3 | 2.69 ± 0.08 | 3 |
| NDGA (Nordihydro- guaiaretic acid) | 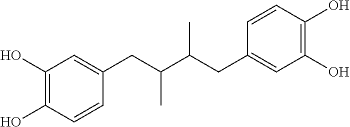 | 1.91 ± 0.02 | 3 | 1.738 ± 0.088 | 3 |
| BML-224 (E)-1-(3,5- Dihydroxyphenyl)- 2-(cyclohexyl) ethene | 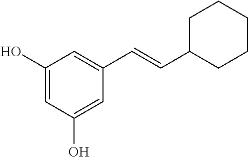 | 1.40 ± 0.23 | 3 | 1.30 ± 0.04 | 3 |
| 3-Hydroxy- trans- stilbene | 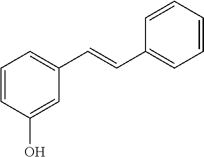 | 0.79 ± 0.06 | 3 | 2.36 ± 0.07 | 3 |
| 4-Methoxy- trans- stilbene | 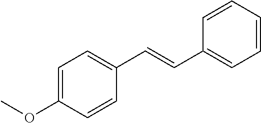 | 0.79 ± 0.20 | 3 | 0.84 ± 0.09 | 3 |
| ZM 336372 | 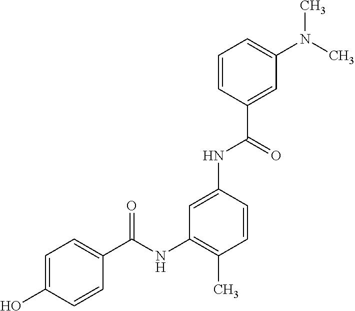 | 0.74 ± 0.05 | 3 | 3.5 ± 1.1 | 3 |
| 3,4- Dihydroxy- trans- stilbene | 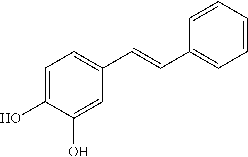 | 0.58 ± 0.25 | 3 | 1.64 ± 0.10 | 6 |

SE stands for standard error of the mean.
N is the number of replicates used to calculate mean ratio to the control rate and standard error.

As can be seen from the results in Table 2, activators of SIRT1 occur in three major groups of polyphenols, namely stilbenes, chalcones and flavones. While SIRT5 is activated to one degree or another by various members of these groups, individual compounds differ significantly in their relative activities with SIRTs 5 and 1. For example, resveratrol, the most potent known natural product activator of SIRT1, and BML-243, a synthetic stilbene somewhat more potent than resveratrol, both are relatively less potent SIRT5 activators. Two other natural stilbenes, piceatannol and pinosylvin are also relatively more potent at activating SIRT1 than SIRT5. Overall, within the stilbene group, SIRT5 displays a marked preference for aliphatic substituents (see Table 2; ethyl: BML-225, isopropyl: BML-231, methyl: BML-228) or halogen substituents (Table 2; chloro: BML-217) in the 4′ position. While stilbene derivatives with these substituents do make good SIRT1 activators, SIRT1 also tolerates a variety of other 4′ substitutions (e.g. hydrogen: pinosylvin, thioethyl: BML-243) that markedly decrease SIRT5 activation. Other notable SIRT5/SIRT1 differences include the strong activation of SIRT5 by non-polyphenol, dipyridamole compounds, and the relatively less potent SIRT5 activation by the chalcones, isoliquiritigenin and butein and by the flavones, fisetin and quercetin.

Thus, in a preferred embodiment, the SIRT5 activating compound of the present invention comprises a polyphenol compound such as a stilbene, chalcone, or flavone or a non-polyphenol dipyridamole, or an analog or derivative thereof. Exemplary SIRT5 activating compounds of the present invention are depicted below in Formulas 1 through 12.

In one embodiment of the present invention, the SIRT5 activating compound comprises a stilbene or chalcone compound of formula 1:

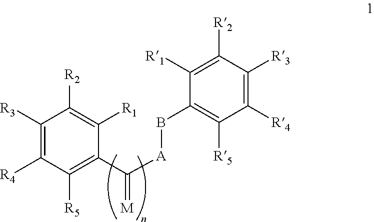

wherein, independently for each occurrence,

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, or carboxyl;

R represents H, alkyl, or aryl;

M represents O, NR, or S;

A-B represents a bivalent alkyl, alkenyl, alkynyl, amido, sulfonamido, diazo, ether, alkylamino, alkylsulfide or hydrazine group, an ethenyl group, or —CH₂CH (Me)CH(Me)CH₂—; and

n is 0 or 1.

In another embodiment, the SIRT5 activating compound comprises a flavanone compound of formula 2:

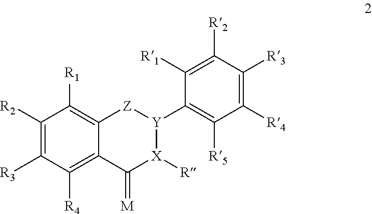

wherein, independently for each occurrence,

R₁, R₂, R₃, R₄, R₅, R′₁, R′₂, R′₃, R′₄, R′₅, and R″ represent H, alkyl, aryl, heteroaryl, alkaryl, heteroaralkyl, halide, NO₂, SR, OR, N(R)₂, or carboxyl;

R represents H, alkyl, or aryl;

M represents H₂, O, NR, or S;

Z represents CR, O, NR, or S; and

X represents CR or N; and

Y represents CR or N.

In another embodiment, the SIRT5 activating compound comprises a flavone compound of formula 3:

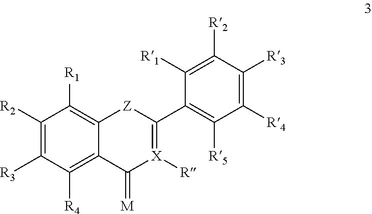

wherein, independently for each occurrence,

R₁, R₂, R₃, R₄, R₅, R′₁, R′₂, R′₃, R′₄, and R′₅, represent H, alkyl, aryl, heteroaryl, alkaryl, heteroaralkyl, halide, NO₂, SR, OR, N(R)₂, or carboxyl;

R″ is absent or represents H, alkyl, aryl, heteroaryl, alkaryl, heteroaralkyl, halide, NO₂, SR, OR, N(R)₂, or carboxyl;

R represents H, alkyl, or aryl;

M represents H₂, O, NR, or S;

Z represents CR, O, NR, or S; and

X represents CR or N when R″ is absent or C when R″ is present.

SIRT5 activating compounds useful in the present invention may also comprise a stilbene, chalcone, or flavone compound represented by formula 4:

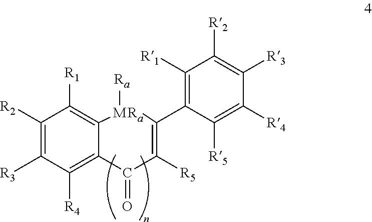

wherein, independently for each occurrence,

M is absent or O;

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, or carboxyl;

Rₐ represents H or the two Rₐ form a bond;

R represents H, alkyl, or aryl; and

n is 0 or 1.

Other SIRT5 activating compounds for use in the present invention include compounds having a formula selected from the group consisting of formulas 5 through 12 set forth below.

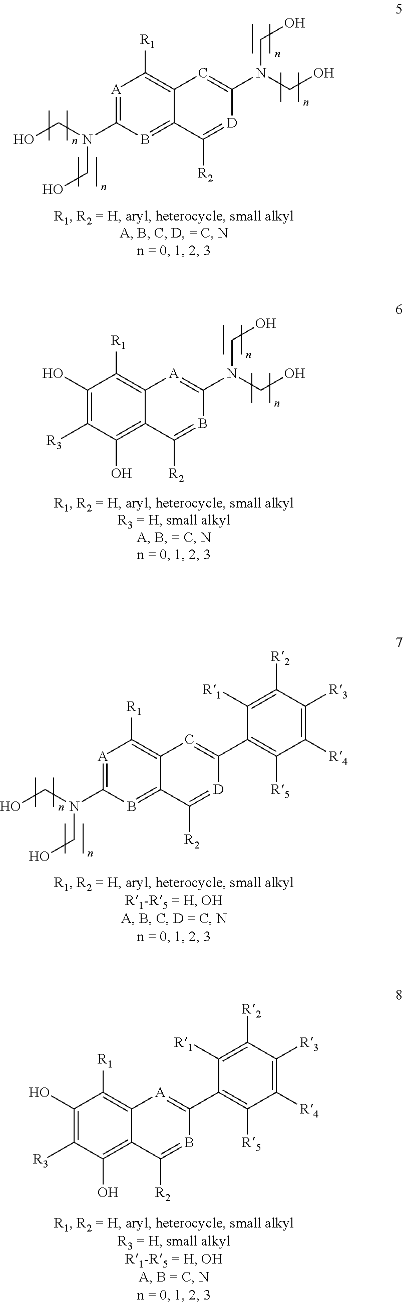

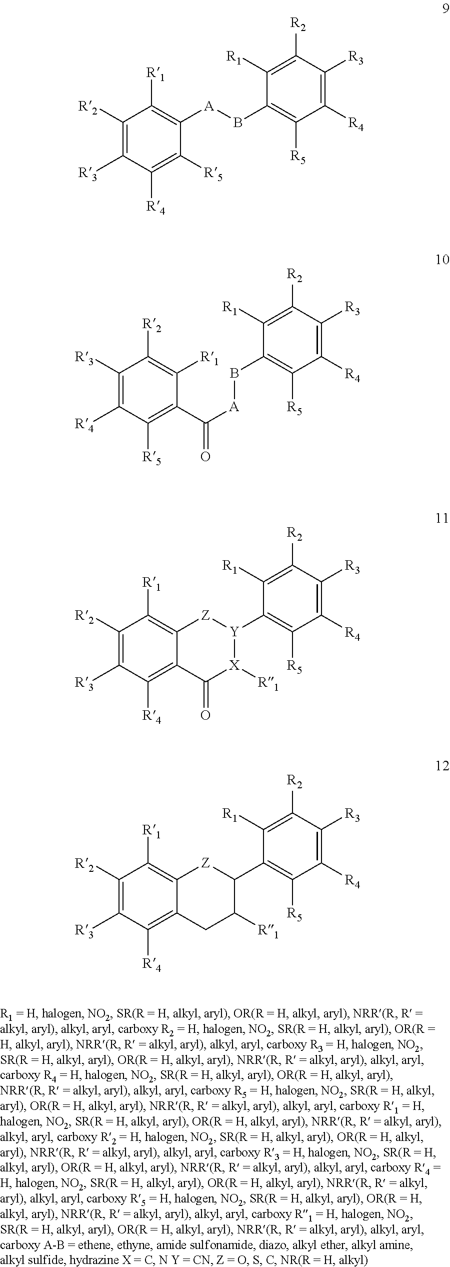

The term “alkyl” is used herein in accordance with its art-recognized meaning and is inclusive of saturated aliphatic groups, including straight-chain alkyl groups, branched-chain alkyl groups, cycloalkyl (alicyclic) groups, alkyl substituted cycloalkyl groups, and cycloalkyl substituted alkyl groups. Straight chain or branched chain alkyls preferably comprise about 30 or fewer carbon atoms in their backbone (e.g., C₁-C₃₀ for straight chain, C₃-C₃₀ for branched chain). Similarly, cycloalkyls have from about 3 to about 10 carbon atoms in their ring structure, more preferably about 5, 6 or 7 carbons in their ring structure. The term “alkyl” is also meant to be inclusive of “substituted alkyls”, meaning alkyl moieties having substituents replacing a hydrogen on one or more carbons of the hydrocarbon backbone. Examples of a substituent include, but are not limited to, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. Further, as will be understood by those skilled in the art upon reading this disclosure, moieties substituted on the hydrocarbon chain may themselves be substituted. For example, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CN and the like. Cycloalkyls may be further substituted with alkyls, alkenyls, alkoxys, alkylthios, aminoalkyls, carbonyl-substituted alkyls, —CN, and the like.

The term “aryl” is also used herein in accordance with its art-recognized meaning and refers to 5-, 6- and 7-membered single-ring aromatic groups that may include from zero to four heteroatoms, for example, benzene, pyrrole, furan, thiophene, imidazole, oxazole, triazole, triazole, pyrazole, pyridine, pyrazine, pyridazine and pyrimidine, and the like. Those aryl groups having heteroatoms in the ring structure may also be referred to as “aryl heterocycles”, “heteroaryls” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The term “aralkyl” is used herein in accordance with its art-recognized meaning and refers to an alkyl group substituted with an aryl group (e.g., an aromatic or heteroaromatic group).

The terms “alkenyl” and “alkynyl” are used herein in accordance with their art-recognized meanings and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “halide”, as used herein, refers to corresponding anions of halogens.

Preferably the SIRT5 activating compound comprises an aliphatically-substituted or halogen-substituted stilbene.

The term “aliphatic” is art-recognized and refers to a linear, branched, cyclic alkane, alkene, or alkyne. Aliphatic substitutions of compounds used in the present invention are linear or branched and have from 1 to about 20 carbon atoms.

Additional exemplary sirtuin activating or inhibiting compounds may be identified in PCT/US2004/021465, the teachings of which are herein incorporated by reference in their entirety.

Preferred exemplary embodiments of human SIRT5 activators for use in the present invention which activate

SIRT5 activity by at least 2-fold as compared to controls include, but are not limited to, 3,5-dihydroxy-4′-chloro-trans-stilbene, dipyridamole, 3,5-dihydroxy-4′ ethyl-trans-stilbene, 3,5-dihydroxy-4′-isopropyl-trans-stilbene, 3,5-dihydroxy-4′-methyl-trans-stilbene, resveratrol, 3,5-dihydroxy-4′ thiomethyl-trans-stilbene, 3,5-dihydroxy-4′-carbomethoxy-trans-stilbene, isoliquiritgenin, 3,5-dihydro-4′ nitro-trans-stilbene, 3,5-dihydroxy-4′ azido-trans-stilbene, piceatannol, 3-methoxy-5-hydroxy-4′ acetamido-trans-stilbene, 3,5-dihydroxy-4′ acetoxy-trans-stilbene, pinosylvin, fisetin, (E)-1-(3,5-dihydrophenyl)-2-(4-pyridyl)ethene, (E)-1-(3,5-dihydrophenyl)-2-(2-napthyl)ethene, 3,5-dihydroxy-4′-acetamide-trans-stilbene, butein, quercetin, 3,5-dihydroxy-4′-thioethyl-trans-stilbene), 3,5-dihydroxy-4′ carboxy-trans-stilbene, and 3,4′-dihydroxy-5-acetoxy-trans-stilbene, and analogs, derivatives or hybrids thereof.

Identified human SIRT5 inhibitors for use in the present invention include, but are not limited to, 3-hydroxy-trans-stilbene, 4-methoxy-trans-stilbene, ZM 336372, and 3,4-dihydroxy-trans-stilbene as depicted in Formulas 13-16, respectively. These compounds are referred to generally herein as human SIRT5 inhibitors or human SIRT5 inhibiting compounds.

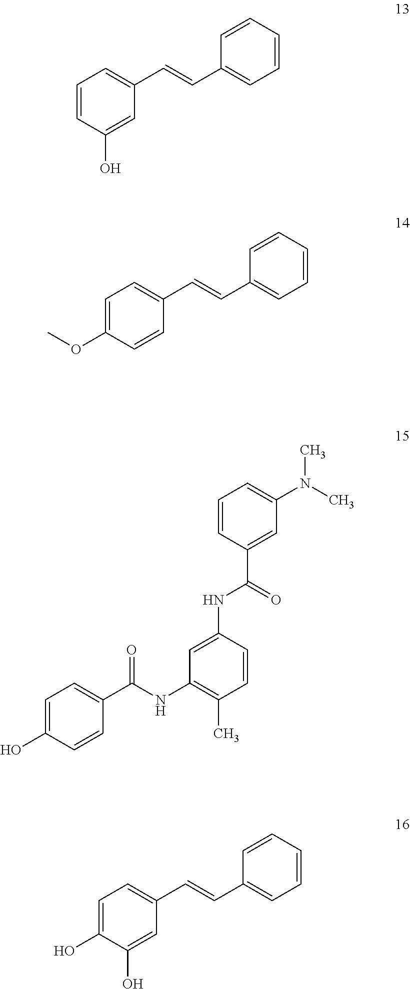

Analogs and derivatives of the above-described compounds of Formulas 1 through 16 can also be used for activating or inhibiting SIRT5. Exemplary derivatives or analogs include, but are not limited to, those making the compounds more stable or improving their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). The resveratrol glucoside, polydatin, also referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Resveratrol and other activating compounds can also be obtained commercially, e.g., from Sigma Chemical Company (St. Louis, Mo.).

In embodiments wherein a compound of Formula 1 through 16 occurs naturally, when used in the present invention, the compound is at least partially isolated from its natural environment prior to use. For example, a plant polyphenol may be isolated from a plant and partially or significantly purified prior to use in the methods described herein. Thus, by isolated, as used herein, it is meant that the compound is preferably associated with less than about 50%, 10%, 1%, 0.1%, 0.01% or 0.001% of a compound with which it is naturally associated.

Compounds for use in the present invention can also be prepared synthetically in accordance with well known methods.

Further compounds of the present invention may be presented in the form of a prodrug releasing the active compound in vivo.

Analysis of the SIRT5 sequence with its positively charged N-terminus and its amphipathic configuration as a helix is indicative of SIRT5 being a mitochondrial transit sequence. Programs based on the correlation of sequence characteristics with subcellular localization predict SIRT5 to be an imported mitochondrial protein (Claros, M. G. and Vincens, Eur. J. Biochem. 241, 779-786 (1996); Emanuelsson, O. et al. J. Mol. Biol. 300, 1005-1016 (2000).

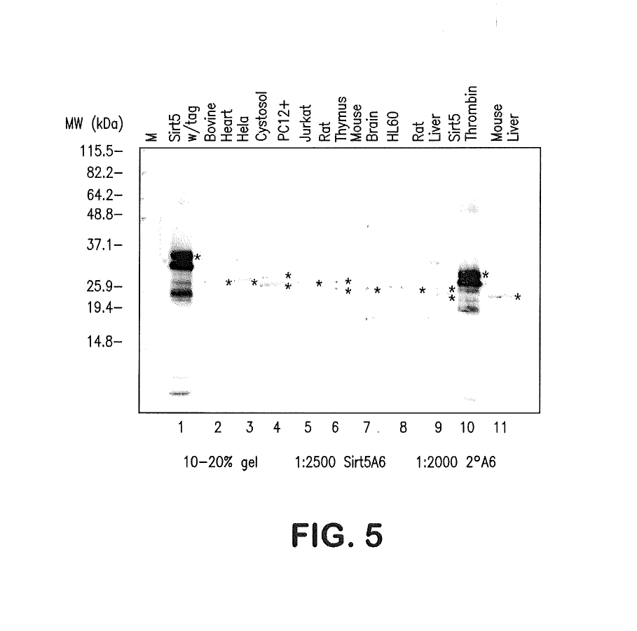

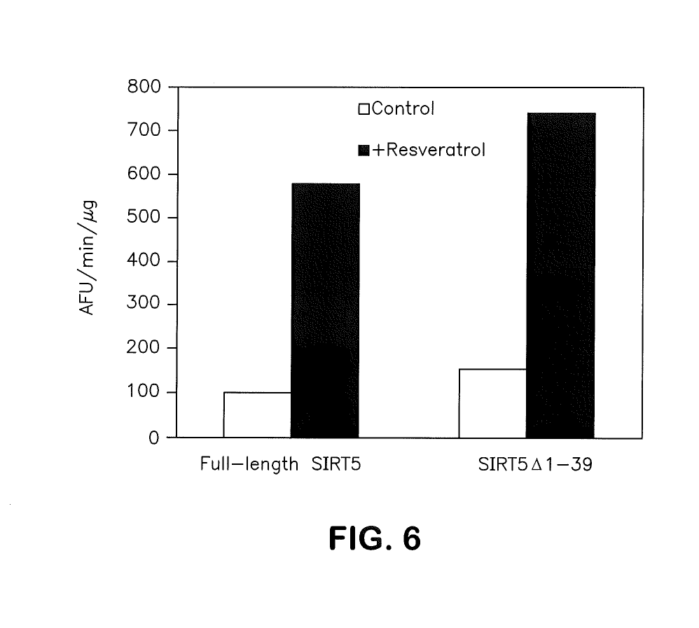

In addition to sequence-based targeting prediction analysis, another line of evidence indicates SIRT5 to be located in the mitochondria. Mitochondrial proteins that, like SIRT5, are encoded in the nucleus and synthesized in the cytoplasm, usually are made as ‘pre-proteins’ containing an N-terminal extension or ‘transit peptide’ which targets the protein to the mitochondria and which is removed by a processing protease upon the protein's import (Hoogenraad, N. J. et al. Biochim. Biophys. Acta 1592, 97-105 (2002); Gakh, O. et al. Biochim. Biophys. Acta 1592, 97-105 (2002)). Since these transit peptides are typically 20-60 amino acids in length (Gakh, O. et al. Biochim. Biophys. Acta 1592, 97-105 (2002)), the mature, imported mitochondrial protein will have a molecular weight that is 2 to 7 kDa less than that of the full-length pre-protein encoded by the nuclear gene. There are two human transcript variants for SIRT5, encoding proteins of 33.9 and 32.7 kDa (Frye, R. A. Biochem. Biophys. Res. Commun. 260, 273-279 (1999); Frye, R. A. Biochem. Biophys. Res. Commun. 273, 793-798 (2000); Genbank Accessions #NM_{—}012241, #NM_{—}031244). The proteins encoded by the bovine (Genbank Accession #NM_{—}583941), mouse (Strausberg, R. L. et al. Proc. Natl. Acad. Sci. U.S.A. 99, 16899-16903 (2002); Genbank Accession #NM_{—}178848) and rat (Strausberg, R. L. et al. Proc. Natl. Acad. Sci. U.S.A. 99, 16899-16903 (2002); Genbank Accession #NM_{—}001004256) SIRT5 transcripts share 85% or greater identity with human and have molecular weights of 34.0, 34.1 and 34.1 kDa, respectively. Further, an antibody prepared against recombinant human SIRT5 has now been found to recognize proteins that range from 25 to 30 kDa in various human and rat cultured cells and mouse, rat and bovine tissues (See FIG. 5). The lower than expected molecular weight of these proteins is consistent with SIRT5 being synthesized as a pre-protein, imported into mitochondria and processed to the lower molecular weight form by removal of an N-terminal transit sequence.

A necessary consequence of SIRT5 being an imported and proteolytically processed mitochondrial protein is that N-terminally truncated SIRT5 should be an active enzyme. Accordingly, a recombinant human SIRT5 (Isoform 1; NM_{—}012241) was constructed in which the first 39 residues were deleted (SIRT5Δ1-39). This produced a protein of 29.6 kDa, which is similar in size to the SIRT5 antibody-reactive bands seen for cultured human Jurkat and HeLa cells (FIG. 5). Tests of the deacetylation activity of SIRT5Δ1-39 with 1 mM of the fluorogenic p53 acetyllysine-382 peptide (BIOMOL Cat. #KI-177) and 12 mM NAD⁺ show the 29.6 kDa protein to be slightly more active than full-length SIRT5 and to be similarly stimulated by 500 μM resveratrol (see FIG. 6). These results demonstrate that when SIRT5 is N-terminally truncated to an extent necessary to produce a protein of a molecular weight similar to that observed in vivo, a modification consistent with a mitochondrial localization, it is an active enzyme and competent to be stimulated by resveratrol.

Further, SIRT5 is a class III sirtuin and therefore a homolog of the CobB bacterial sirtuins, which have been shown to catalyze the regulatory (activating) deacetylation acetyl-CoA synthetases (Starai, V. J. et al. Science 298, 2390-2392 (2002); Zhao, K. et al. J. Mol. Biol. 337, 731-741 (2004)). These enzymes catalyze the ligation of acetate and CoA, at the expense of the formation of AMP and pyrophosphate from ATP.

In mammals, free acetate is derived from various sources including ethanol metabolism, the action of bacteria in the gut, and the hydrolysis of acetyl-CoA by the enzyme acetyl-CoA hydrolase (Crabtree, B. et al. Biochem. J. 257, 673-678 (1989); Akanji, A. O. et al. Clin. Chim. Acta 185, 25-34 (1989)). Plasma acetate levels are elevated by ketogenic conditions such as starvation and by type 2 diabetes (Akanji, A. O. et al. Clin. Chim. Acta 185, 25-34 (1989); Buckley, B. M. and Williamson, D. H. Biochem. J. 166, 539-545 (1977)). There are two known human acetyl-CoA synthetases, one cytoplasmic (AceS1; Luong, A. et al. J. Biol. Chem. 275, 26458-26466 (2000)) and the other mitochondrial (AceS2; Fujino, T. et al. J. Biol. Chem. 276, 11420-11426 (2001)). Both include the highly conserved motif which surrounds the acetylation site in the bacterial acetyl-CoA synthetases (Luong, A. et al. J. Biol. Chem. 275, 26458-26466 (2000); Fujino, T. et al. J. Biol. Chem. 276, 11420-11426 (2001); Starai, V. J. et al. Science 298, 2390-2392 (2002)). Expression of AceS1 is negatively regulated by sterols by way of the action of sterol regulatory element-binding proteins (SREBPs; Luong, A. et al. J. Biol. Chem. 275, 26458-26466 (2000)). Elevation of AceS1 activity promotes incorporation of acetate into lipids and cholesterol (Luong, A. et al. J. Biol. Chem. 275, 26458-26466 (2000)). In contrast, an increase in the mitochondrial AceS2 activity directs acetate primarily towards oxidation and energy production (Fujino, T. et al. J. Biol. Chem. 276, 11420-11426 (2001)).

The relationship between S1RT5 and AceS2 is indicative of SIRT5 activating compounds being useful in modulating, and more specifically activating mitochondrial AceS2. SIRT5 activating compounds may prove to be useful lipid-lowering agents. Such agents are expected to be particularly useful in conditions such as type 2 diabetes, in which acetate levels are elevated and an increase in AceS2 activity can divert the acetate pool towards oxidation and thereby away from AceS1 and consequent lipid and cholesterol synthesis. Given that hyperlipidemia and hyper-cholesterolemia are implicated, respectively, in the pathogenesis (Biden, T. J. et al. Diabetes 53 (Suppl. 1) S159-S165 (2004)) and complications (Snow, V. et al. Ann. Intern. Med. 140, 644-649 (2004)) of type 2 diabetes, SIRT5-activating agents of the present invention are expected to be of benefit to both prevention and treatment of this disease.

Although much medical attention has focused on its dangers, cholesterol is necessary as a structural component of cell membranes and as a precursor of steroid hormones and bile acids. Hypocholesterolemia is associated with a number of pathologic states, including traumatic injury (Dunham, C. M. et al. Crit. Care Med. 22, 667-672 (1994)), sepsis (Alvarez, C. and Ramos, A. Clin. Chem. 32, 142-145 (1986)) and sickle cell anemia (VanderJagt, D. J. et al. J. Trop. Pediatr. 48, 156-161 (2002)). Hypocholesterolemia is predictive of increased mortality in critically ill surgical patients (Gordon, B. R. et al. Crit. Care Med. 29, 1563-1568 (2001)), patients with multiple organ failure (Fraunberger, P. et al. Crit. Care Med. 28, 3574-3575 (2000)) and patients on maintenance kidney dialysis (Kalantar-Zadeh, K. et al. Kidney Int. 63, 793-808 (2003)). The mitochondrial localization for SIRT5 and its potential role in activation of AceS2 is indicative of SIRT inhibitors raising cholesterol and lipid levels thereby preventing diversion of acetate away from AceS1 and the synthetic pathway. Thus, it is believed that SIRT5 inhibitors may be useful in the treatment of illnesses and/or conditions associated with hypocholesterolemia including, but not limited to, traumatic injury, sickle cell anemia, multiple organ failure and kidney dialysis.

Further, although the mitochondria are considered the most probable location for SIRT5, this enzyme could be localized in the cytoplasm. If localized in the cytoplasm, AceS1 rather than AceS2 is the likely target for deacetylation and activation by SIRT5. In this case, the pharmacological uses of SIRT5 activators and inhibitors would be reversed; i.e. activators would be useful in raising cholesterol and lipid levels and inhibitors would be useful in lowering them.

SIRT5 activating and inhibiting compounds useful in the methods of the present invention may be formulated for administration in any suitable manner. They may, for example, be formulated for topical administration or administration by inhalation or, more preferably, for oral, transdermal or parenteral administration. The pharmaceutical composition may be in a form such that it can effect controlled release of the SIRT5 activating or inhibiting compound. A particularly preferred method of administration, and corresponding formulation, is oral administration.

For oral administration, the pharmaceutical composition may take the form of, and be administered as, for example, tablets (including sub-lingual tablets) and capsules (each including timed release and sustained release formulations), pills, powders, granules, elixirs, tinctures, emulsions, solutions, syrups or suspensions prepared by conventional means with acceptable excipients.

For instance, for oral administration in the form of a tablet or capsule, the SIRT5 activating or inhibiting compound can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agents can also be present.

Capsules can be made by preparing a powder mixture as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. SIRT5 activating or inhibiting compounds useful in the methods of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or saccharin, and the like can also be added.

Where appropriate, dosage unit formulations for oral administration can be microencapsulated. The formulation can also be prepared to prolong or sustain the release as for example by coating or embedding particulate material in polymers, wax or the like.

SIRT5 activating or inhibiting compounds for use in the methods of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

SIRT5 activating or inhibiting compounds for use in the methods of the present invention can also be administered in the form of liposome emulsion delivery systems, such as small unilamellar vesicles, large unilamellar vesicles and multilamellar vesicles. Liposomes can be formed from a variety of phospholipids, such as cholesterol, stearylamine or phosphatidylcholines.

The present invention includes pharmaceutical compositions containing 0.1 to 99.5%, more particularly, 0.5 to 90% of a SIRT5 activating or inhibiting compound in combination with a pharmaceutically acceptable carrier.

Compositions comprising a SIRT5 activating or inhibiting compound may also be administered in nasal, ophthalmic, otic, rectal, topical, intravenous (both bolus and infusion), intraperitoneal, intraarticular, subcutaneous or intramuscular inhalation or insufflation form, all using forms well known to those of ordinary skill in the pharmaceutical arts.

For transdermal administration, the pharmaceutical composition comprising the SIRT5 activating or inhibiting compound may be given in the form of a transdermal patch, such as a transdermal iontophoretic patch.

For parenteral administration, the pharmaceutical composition comprising the SIRT5 activating or inhibiting compound may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilizing and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

SIRT5 activating or inhibiting compound for use in the methods of the present invention may also be formulated as a depot preparation. Such long acting formulations may be administered by implantation (for example subcutaneously or intramuscularly) or by intramuscular injection. Thus, for example, the SIRT5 activating or inhibiting compound may be formulated with suitable polymeric or hydrophobic materials (for example as an emulsion in an acceptable oil) or ion exchange resins, or as sparingly soluble derivatives, for example, as a sparingly soluble salt.

Alternatively the SIRT5 activating or inhibiting compound may be formulated for topical application, for example in the form of ointments, creams, lotions, eye ointments, eye drops, ear drops, mouthwash, impregnated dressings and sutures and aerosols, and may contain appropriate conventional additives, including, for example, preservatives, solvents to assist drug penetration, and emollients in ointments and creams. Such topical formulations may also contain compatible conventional carriers, for example cream or ointment bases, and ethanol or oleyl alcohol for lotions. Such carriers may constitute from about 1% to about 98% by weight of the formulation; more usually they will constitute up to about 80% by weight of the formulation.

For administration by inhalation the SIRT5 activating or inhibiting compound can be conveniently delivered in the form of an aerosol spray presentation from pressurized packs or a nebulizer, with the use of a suitable propellant, e.g. dichlorodifluoromethane, trichlorofluoromethane, dichlorotetrafluoroethane, tetrafluoroethane, heptafluoropropane, carbon dioxide or other suitable gas. In the case of a pressurized aerosol, the dosage unit may be determined by providing a valve to deliver a metered amount. Capsules and cartridges of, for example, gelatin, for use in an inhaler or insufflator may be formulated containing a powder mix of a SIRT5 activating or inhibiting compound and a suitable powder base such as lactose or starch.

Pharmaceutical compositions comprising a SIRT5 activating compound are administered in an amount effective at activating mitochondrial AceS2 to divert the acetate pool towards oxidation and thereby away from AceS1 and consequent lipid and cholesterol synthesis. These agents are expected to useful as lipid-lowering agents, particularly in the prevention and treatment of type 2 diabetes. Initial dosing in humans is accompanied by clinical monitoring of symptoms for such conditions. In general, the compositions are administered in an amount of active agent of at least about 100 μg/kg body weight. In most cases they will be administered in one or more doses in an amount not in excess of about 20 mg/kg body weight per day. Preferably, in most cases, dose is from about 100 μg/kg to about 5 mg/kg body weight, daily. For administration particularly to mammals, and particularly humans, it is expected that the daily dosage level of the active agent will be from 0.1 mg/kg to 10 mg/kg and typically around 1 mg/kg. It will be appreciated that optimum dosage will be determined by standard methods for each treatment modality and indication, taking into account the indication, its severity, route of administration, complicating conditions and the like. The physician in any event will determine the actual dosage that will be most suitable for an individual and will vary with the age, weight and response of the particular individual. The effectiveness of a selected actual dose can readily be determined, for example, by measuring clinical symptoms or standard indicia of hyperlipidemia and/or hypercholsteremia, particularly when associated with type 2 diabetes after administration of the selected dose. The above dosages are exemplary of the average case. There can, of course, be individual instances where higher or lower dosage ranges are merited, and such are within the scope of this invention. For conditions or disease states as are treated by the present invention, maintaining consistent daily levels in a subject over an extended period of time, e.g., in a maintenance regime, can be particularly beneficial.

As shown by Table 2, some of the test compounds activate the two sirtuins, namely SIRT5 and SIRT1 similarly, while others activate them differentially. Thus, as shown in Table 2, compounds may be selective activators or inhibitors of human SIRT5 or SIRT1, or alternatively general activators or inhibitors of sirtuins including, but not limited to, human SIRT5 and human SIRT1. For example, dipyridamole and BML-237 (3,5-dihydroxy-4′-carbomethoxy-trans-stilbene) are identified herein as selective activators of SIRT5 as compared to SIRT1; BML-217 (3,5-dihydroxy-4′-chloro-trans-stilbene) is identified herein as a potent activator of SIRT5 and SIRT1; and BML-243, butein and ZM336372 are identified herein as selective activators of SIRT1 as compared to SIRT5.

Accordingly, the present invention also relates to method for identifying selective activators or inhibitors of human SIRT1 or human SIRT5 activity and using such compounds to selectively activate or inhibit human SIRT1 or human SIRT5, respectively. Selective activators of human SIRT1 are expected to be useful in modulating p53 acetylation and apoptosis and extending the lifespan of a eukaryotic cells and/or increasing their resistance to stress, while selective activators or inhibitors of SIRT 5 are expected to be useful in modulating mitochondrial AceS2, lowering lipid levels and preventing and/or treating type 2 diabetes.

Assays to identify selective SIRT1 or SIRT5 activating or inhibiting compounds versus general activators or inhibitors of sirtuins may be conducted in a cell based or cell free format. For example, an assay may comprise incubating (or contacting) a selected sirtuin, preferably SIRT1 or SIRT5 with a test compound under conditions in which the SIRT1 or SIRT5 can be activated by an agent known to activate the SIRT1 or SIRT5, and monitoring or determining the level of activation of the SIRT1 or SIRT5 in the presence of the test compound relative to the absence of the test compound. The level of activation of SIRT1 or SIRT5 can be determined by determining its ability to deacetylate a substrate. Exemplary substrates are acetylated peptides, e.g., those set forth herein in Table 1. A particularly preferred substrate is the Fluor de Lys-SIRT1 (BIOMOL Cat. #KI-177), i.e., the acetylated peptide Arg-His-Lys-Lys(Ac) (SEQ ID NO:32). Other substrates are peptides from human histones H3 and H4 or an acetylated amino acid. Substrates may be fluorogenic. The sirtuin may be SIRT1 or SIRT5 or a portion thereof. For example, recombinant SIRT1 can be obtained from BIOMOL. The reaction may be conducted for about 30 minutes and stopped, e.g., with nicotinamide. The HDAC fluorescent activity assay/drug discovery kit (AK-500, BIOMOL Research Laboratories) may be used to determine the level of acetylation. Similar assays are described in Bitterman et al. (2002) J. Biol. Chem. 277:45099. The level of activation of the SIRT1 or SIRT5 in an assay may be compared to the level of activation of the SIRT1 or SIRT5 in the presence of one or more (separately or simultaneously) compounds described herein, which may serve as positive or negative controls. In addition, the activity of the compound in the presence of SIRT1 can be compared to the activity of the compound in the presence of SIRT5 and vice versa. It has been shown herein that activating compounds appear to interact with the N-terminus of SIRT1. Accordingly, full length sirtuin proteins or portions of the sirtuin proteins inclusive of the N-terminal portions of sirtuins, e.g., about amino acids 1-176 or 1-255 of SIRT1; about amino acids.

In one embodiment, a screening assay comprises first contacting SIRT1 with a test compound and an acetylated substrate under conditions appropriate for the SIRT1 to deacetylate the substrate in the absence of the test compound and determining the level of deacetylation of the substrate by SIRT1 in the presence of the test compound. SIRT5 is then contacted with the same test compound and the same acetylated substrate under the same conditions used to measure deacetylation by SIRT1 and the level of deacetylation of the substrate by SIRT5 in the presence of the test compound is determined. The deacetylation levels of the substrate by SIRT1 versus SIRT5 are then compared. Higher levels of deacetylation of the substrate by SIRT1 as compared to SIRT5 is indicative of the test compound being a selective SIRT1 activating compound. Higher levels of deacetylation of the substrate by SIRT5 as compared to SIRT1 is indicative of the test compound being a selective SIRT5 activating compound. Equal levels of deacetylation of the substrate by SIRT1 and SIRT5 is indicative of the test compound being a general activator of sirtuins.

Western blotting, preferably combined with cell fractionation is also expected to provide a useful assay for measuring SIRT1 versus SIRT selectivity.

Further, to date, no class Ia sirtuin has had its structure solved. On the other hand structures have been determined for three class III enzymes, namely E. coli CobB (Zhao, K. et al. J. Mol. Biol. 337, 731-741 (2004)) and both A. fulgidus sirtuins, Sir2-Af1 (Min, J. et al. Cell 105, 269-279 (2001)) and Sir2-Af2 (Avalos, J. L. Mol. Cell. 10, 523-535 (2002)). Thus, identification herein of structurally defined class III enzymes having overlapping patterns of activation with class Ia enzymes provides a useful tool for identifying not only class III activators and inhibitors but also class Ia activators and inhibitors. For example, SIRT5 can be co-crystallized with one of a SIRT5 activating compound such as identified herein and the three-dimensional structure of the complex can be determined. Information relating to the interactions between the SIRT5 activating compound and SIRT5 residues and/or the shape of the activator binding site can then be entered into computer modeling programs to design new, and potentially more potent, activators of SIRT5 and/or SIRT1. As will be understood by one of skill in the art upon reading this disclosure, SIRT5 and/or SIRT1 inhibiting compounds can be designed in a similar manner.

The following nonlimiting examples are provided to further illustrate the present invention

### EXAMPLES

### Example 1

### SIRT5 Deactylation Assay

In these experiments, SIRT5 (26.5 μg in total volume 50 μl) was incubated at 37° C. for 71.5 minutes in the presence of 500 μM of the indicated peptides plus 500 μM NAD⁺ in sirtuin assay buffer (BIOMOL Cat. #, 25 mM Tris/C1, pH 8.0, 137 mM NaCl, 2.7 mM KCl, 1 mM MgCl₂, 1 mg/ml BSA). Reactions were terminated and the extent of deacetylation determined by addition of 50 μl “Fluor de Lys Developer II” (BIOMOL Cat. #KI-176) plus 2 mM nicotinamide. After 45 minutes, the resulting fluorescence was read in ½-volume 96-well white microplates (BIOMOL Cat. #KI-110) with a CytoFluor™.II fluorescence plate reader (PerSeptive Biosystems) at an excitation wavelength of 360 nm, an emission wavelength of 460 nm and a gain of 85. Results are represented as Arbitrary Fluorescence Units or AFUs.

### Example 2

### SIRT5 Rate Measurement

All SIRT5 rate measurements used in the calculation of “Ratio to Control Rate” were obtained with 100 μM NAD⁺ and 500 μM p53-382 acetylated peptide substrate, but otherwise were performed as described K. T. Howitz et al. (Nature 2003 425 191). All ratio data were calculated from experiments in which the total deacetylation in the control reaction was less than 1% of the initial concentration of acetylated peptide.

### Example 3

### SIRT1 Rate Measurement

All SIRT1 rate measurements used in the calculation of “Ratio to Control Rate” were obtained with 25 μM NAD⁺ and 25 μM p53-382 acetylated peptide substrate were performed as described in by K. T. Howitz et al. (Nature 2003 425 191). All ratio data were calculated from experiments in which the total deacetylation in the control reaction was 0.25-1.25 μM peptide or 1-5% of the initial concentration of acetylated peptide.

What is claimed is:

1. A method for modulating human SIRT5 activity comprising contacting human SIRT5 with a polyphenol compound or an analog or derivative thereof selected from the group consisting of stilbenes, chalcones, and flavones, or a non-polyphenol dipyridamole compound.

2. The method of claim 1 wherein human SIRT5 is activated and the polyphenol compound or non-polyphenol dipyridamole compound comprises a compound selected from Formula 1, 2, 3, 4, 5, 6, 7, 8, 9, 10, 11 and 12.

3. The method of claim 2 wherein the polyphenol compound or non-polyphenol dipyridamole compound is selected from the group consisting of 3,5-dihydroxy-4′-chloro-trans-stilbene, dipyridamole, 3,5-dihydroxy-4′ ethyl-trans-stilbene, 3,5-dihydroxy-4′-isopropyl-trans-stilbene, 3,5-dihydroxy-4′-methyl-trans-stilbene, resveratrol, 3,5-dihydroxy-4′ thiomethyl-trans-stilbene, 3,5-dihydroxy-4′-carbomethoxy-trans-stilbene, isoliquiritgenin, 3,5-dihydro-4′ nitro-trans-stilbene, 3,5-dihydroxy-4′ azido-trans-stilbene, piceatannol, 3-methoxy-5-hydroxy-4′ acetamido-trans-stilbene, 3,5-dihydroxy-4′ acetoxy-trans-stilbene, pinosylvin, fisetin, (E)-1-(3,5-dihydrophenyl)-2-(4-pyridyl)ethene, (E)-1-(3,5-dihydrophenyl)-2-(2-napthyl)ethene, 3,5-dihydroxy-4′-acetamide-trans-stilbene, butein, quercetin, 3,5-dihydroxy-4′-thioethyl-trans-stilbene), 3,5-dihydroxy-4′ carboxy-trans-stilbene, and 3,4′-dihydroxy-5-acetoxy-trans-stilbene, or an analog or derivative thereof.

4. The method of claim 1 wherein human SIRT5 is inhibited and the polyphenol compound or non-polyphenol dipyridamole compound is selected from the group consisting of 3-hydroxy-trans-stilbene, 4-methoxy-trans-stilbene, ZM 336372, and 3,4-dihydroxy-trans-stilbene.

5. A method for modulating mitochondrial acetyl-CoA synthetase (AceS2) activity in cells comprising contacting cells with a polyphenol compound selected from the group consisting of stilbenes, chalcones, and flavones or a non-polyphenol dipyridamole compound, or an analog or derivative thereof.

6. The method of claim 5 wherein mitochondrial acetyl-CoA synthetase (AceS2) is activated and the polyphenol compound or non-polyphenol dipyridamole compound comprises a compound selected from Formula 1, 2, 3, 4, 5, 6, 7, 8, 9, 10, 11 and 12.

7. The method of claim 6 wherein the polyphenol compound or non-polyphenol dipyridamole compound is selected from the group consisting of 3,5-dihydroxy-4′-chloro-trans-stilbene, dipyridamole, 3,5-dihydroxy-4′ ethyl-trans-stilbene, 3,5-dihydroxy-4′-isopropyl-trans-stilbene, 3,5-dihydroxy-4′-methyl-trans-stilbene, resveratrol, 3,5-dihydroxy-4′ thiomethyl-trans-stilbene, 3,5-dihydroxy-4′-carbomethoxy-trans-stilbene, isoliquiritgenin, 3,5-dihydro-4′ nitro-trans-stilbene, 3,5-dihydroxy-4′ azido-trans-stilbene, piceatannol, 3-methoxy-5-hydroxy-4′ acetamido-trans-stilbene, 3,5-dihydroxy-4′ acetoxy-trans-stilbene, pinosylvin, fisetin, (E)-1-(3,5-dihydrophenyl)-2-(4-pyridyl)ethene, (E)-1-(3,5-dihydrophenyl)-2-(2-napthyl)ethene, 3,5-dihydroxy-4′-acetamide-trans-stilbene, butein, quercetin, 3,5-dihydroxy-4′-thioethyl-trans-stilbene), 3,5-dihydroxy-4′ carboxy-trans-stilbene, and 3,4′-dihydroxy-5-acetoxy-trans-stilbene, or an analog or derivative, thereof.

8. The method of claim 5 wherein mitochondrial acetyl-CoA synthetase (AceS2) is inhibited and the polyphenol compound or non-polyphenol dipyridamole compound is selected from the group consisting of 3-hydroxy-trans-stilbene, 4-methoxy-trans-stilbene, ZM 336372, and 3,4-dihydroxy-trans-stilbene.

9. A method for lowering lipids in a subject comprising administering to the subject a pharmaceutical composition comprising a polyphenol compound selected from the group consisting of stilbenes, chalcones or flavones or a non-polyphenol dipyridamole compound or an analog or derivative thereof and a pharmaceutically acceptable carrier.

10. The method of claim 9 wherein the polyphenol compound or non-polyphenol dipyridamole compound is selected from Formula 1, 2, 3, 4, 5, 6, 7, 8, 9, 10, 11 and 12.

11. The method of claim 10 wherein the polyphenol compound or non-polyphenol dipyridamole compound is selected from the group consisting of 3,5-dihydroxy-4′-chloro-trans-stilbene, dipyridamole, 3,5-dihydroxy-4′ ethyl-trans-stilbene, 3,5-dihydroxy-4′-isopropyl-trans-stilbene, 3,5-dihydroxy-4′-methyl-trans-stilbene, resveratrol, 3,5-dihydroxy-4′ thiomethyl-trans-stilbene, 3,5-dihydroxy-4′-carbomethoxy-trans-stilbene, isoliquiritgenin, 3,5-dihydro-4′ nitro-trans-stilbene, 3,5-dihydroxy-4′ azido-trans-stilbene, piceatannol, 3-methoxy-5-hydroxy-4′ acetamido-trans-stilbene, 3,5-dihydroxy-4′ acetoxy-trans-stilbene, pinosylvin, fisetin, (E)-1-(3,5-dihydrophenyl)-2-(4-pyridyl)ethene, (E)-1-(3,5-dihydrophenyl)-2-(2-napthyl)ethene, 3,5-dihydroxy-4′-acetamide-trans-stilbene, butein, quercetin, 3,5-dihydroxy-4′-thioethyl-trans-stilbene), 3,5-dihydroxy-4′ carboxy-trans-stilbene, and 3,4′-dihydroxy-5-acetoxy-trans-stilbene, or an analog or derivative thereof.

12. A method for treating or preventing with hyperlipidemia, hypercholesterolemia or type 2 diabetes in a patient comprising administering to the patient a pharmaceutical composition comprising a polyphenol compound selected from the group consisting of stilbenes, chalcones, and flavones or a non-polyphenol dipyridamole compound, or an analog or derivative thereof and a pharmaceutically acceptable carrier.

13. The method of claim 12 wherein the polyphenol compound or non-polyphenol dipyridamole compound is selected from Formula 1, 2, 3, 4, 5, 6, 7, 8, 9, 10, 11 and 12.

14. The method of claim 13 wherein the polyphenol compound or non-polyphenol dipyridamole compound is selected from the group consisting of 3,5-dihydroxy-4′-chloro-trans-stilbene, dipyridamole, 3,5-dihydroxy-4′ ethyl-trans-stilbene, 3,5-dihydroxy-4′-isopropyl-trans-stilbene, 3,5-dihydroxy-4′-methyl-trans-stilbene, resveratrol, 3,5-dihydroxy-4′ thiomethyl-trans-stilbene, 3,5-dihydroxy-4′-carbomethoxy-trans-stilbene, isoliquiritgenin, 3,5-dihydro-4′ nitro-trans-stilbene, 3,5-dihydroxy-4′ azido-trans-stilbene, piceatannol, 3-methoxy-5-hydroxy-4′ acetamido-trans-stilbene, 3,5-dihydroxy-4′ acetoxy-trans-stilbene, pinosylvin, fisetin, (E)-1-(3,5-dihydrophenyl)-2-(4-pyridyl)ethene, (E)-1-(3,5-dihydrophenyl)-2-(2-napthyl)ethene, 3,5-dihydroxy-4′-acetamide-trans-stilbene, butein, quercetin, 3,5-dihydroxy-4′-thioethyl-trans-stilbene), 3,5-dihydroxy-4′ carboxy-trans-stilbene, and 3,4′-dihydroxy-5-acetoxy-trans-stilbene, or an analog or derivative thereof.

15. A method for treating or preventing with hypocholesterolemia in a patient comprising administering to the patient a pharmaceutical composition comprising a polyphenol compound or non-polyphenol dipyridamole selected from the group consisting of 3-hydroxy-trans-stilbene, 4-methoxy-trans-stilbene, ZM 336372, and 3,4-dihydroxy-trans-stilbene.

16. A method of detecting a modulator of SIRT5 activity comprising contacting SIRT5 with a test compound under conditions in which SIRT5 is activated by an agent known to activate SIRT5 and monitoring or determining the level of activity of the SIRT5 in the presence of the test compound relative to the level of activity of the SIRT5 in the absence of the test compound, wherein the compound is a SIRT5 activator if the level of SIRT5 activity in the presence of the test compound is greater than the level of SIRT5 activity in the absence of the test compound, and the compound is a SIRT5 inhibitor if the level of SIRT5 activity in the presence of the test compound is less than the level of SIRT5 activity in the absence of the test compound.

17. The method of claim 16, wherein the SIRT 5 comprises an N-terminal deletion of amino acids 1-39 or a portion thereof.

18. The method of claim 16, wherein the SIRT5 includes its N-terminal portion.

19. The method of claim 16, wherein the level of activity of SIRT5 is determined by deacetylation of a substrate.

20. The method of claim 19, wherein the substrate is an acetylated peptide.

21. The method of claim 20, wherein the acetylated peptide is SEQ ID NO:25, SEQ ID NO:26, SEQ ID NO:27, SEQ ID NO:28, SEQ ID NO:29, SEQ ID NO:30, SEQ ID NO:31 or SEQ ID NO:32.

22. The method of claim 20, wherein the acetylated peptide is SEQ ID NO: 26.

23. The method of claim 19, wherein the substrate is fluorogenic.

24. The method of claim 16, wherein SIRT5 is contacted with the test compound under conditions in which SIRT5 activity is activated by an agent known to activate SIRT5 deacetylation activity at a concentration of about 200 μM of the agent using an acetylated peptide substrate at a concentration of about 100 μM for about 30 minutes prior to stopping the reaction.

25. The method of claim 24, wherein the reaction conditions comprise a NAD⁺ substrate concentration of about 500 μM.

26. The method of claim 24, wherein the SIRT5 reaction is stopped by addition of nicotinamide.

27. The method of claim 16, wherein the level of acetylation is determined by a histone deactylase fluorescent activity assay.

28. The method of claim 16, wherein the SIRT5 activity is monitored or determined in a cell based format.

29. The method of claim 16, wherein the SIRT5 reaction is monitored or determined in a cell free format.

30. The method of claim 16, further comprising detecting the test compound's ability to modulate SIRT1, wherein if the test compound modulates the activity of SIRT5 but not SIRT1 then the test compound is a SIRT5-specific sirtuin modulator, and if the test compound modulates the activity of SIRT1 but not SIRT5 then the compound is a SIRT1-specific sirtuin modulator.

31. A method for identifying compounds as activators of human SIRT5 or human SIRT1 or general activators of SIRT5 and SIRT1 comprising:
(i) contacting SIRT1 with a test compound and an acetylated substrate under conditions appropriate for the SIRT1 to deacetylate the substrate in the absence of the test compound;
(ii) determining the level of deacetylation of the substrate by SIRT1 in the presence of the test compound,
(iii) contacting SIRT5 with the same test compound and the same acetylated substrate under the same conditions used in step (i) for the SIRT1;
(iv) determining the level of deacetylation of the substrate by SIRT5 in the presence of the test compound; and (v) comparing deacetylation levels of the substrate determined in steps (ii) and (iv) wherein a higher level of deacetylation of the substrate by SIRT1 as compared to SIRT5 is indicative of the test compound being a SIRT1 activating compound, wherein a higher level of deacetylation of the substrate by SIRT5 as compared to SIRT1 is indicative of the test compound being a SIRT5-specific activating compound, and wherein equal levels of deactylation of the substrate by SIRT1 and SIRT5 is indicative of the test compound being a general activator of SIRT1 and SIRT5.

32. A method for selectively activating human SIRT1 activity by contacting SIRT1 with a compound identified in accordance with the method of claim 31 to selectively activate human SIRT1 as compared to human SIRT5.

33. The method of claim 32 wherein the compound is BML-243, butein or ZM336372.

34. A method for selectively activating human SIRT5 activity by contacting SIRT5 with a compound identified in accordance with the method of claim 31 to selectively activate human SIRT1 as compared to human SIRT5.

35. The method of claim 34 wherein the compound is dipyridamole or BML-237 (3,5-dihydroxy-4′-carbomethoxy-trans-stilbene).
